(19) United States
(12) Patent Application Publication
(10) Pub. No.: US 2022/0364075 A1
(43) Pub. Date: Nov. 17, 2022

(54) METHODS OF MODIFYING A NUCLEIC ACID SEQUENCE

(71) Applicant: FLAGSHIP PIONEERING, INC., Cambridge, MA (US)

(72) Inventors: Christine Elizabeth Hajdin, Boston, MA (US); David Arthur Berry, Newton, MA (US); Theonie Anastassiadis, Boston, MA (US); Noubar Boghos Afeyan, Lexington, MA (US)

(21) Appl. No.: 17/774,411

(22) PCT Filed: Nov. 4, 2020
(86) PCT No.: PCT/US2020/058958

§ 371 Date: May 4, 2022

Related U.S. Application Data
(60) Provisional application No. 62/930,367, filed on Nov. 4, 2019.

(51) Int. Cl. C12N 15/10; C12Q 1/6869; C12N 15/67; C12Q 1/6806

(52) U.S. Cl. CPC C12N 15/102; C12Q 1/6869; C12N 15/67; C12Q 1/6806

(57) ABSTRACT

The disclosure relates generally to methods of modifying a nucleic acid sequence, systems for modifying a nucleic acid sequence, and compositions made by said methods or systems.

### CROSS-REFERENCE TO RELATED APPLICATIONS

This application claims priority to U.S. Provisional Application 62/930,367 filed on Nov. 4, 2019, the entire contents of which is hereby incorporated by reference.

### BACKGROUND

Due to the degeneracy of the genetic code, most amino acids can be encoded by more than one trinucleotide codon. Furthermore, codon usage can differ between organisms. This allows for the manipulation of nucleic acid sequences encoding an amino acid sequence to alter protein expression—conventionally also referred to as codon optimization.

### SUMMARY

The inventors have discovered that the codon identity and function is in fact highly contextual and varied within an organism. In one aspect, the disclosure provides a method of modifying, e.g., contextually modifying, a nucleic acid sequence, e.g., a nucleic acid sequence comprising an open reading frame (ORF) or coding sequence (CDS), for expression in a target cell or tissue comprising: a) acquiring the identity of a contextually rare codon (“con-rare codon”) for the nucleic acid sequence in the target cell or tissue; b)(i) replacing a con-rare codon in the nucleic acid sequence with a contextually abundant (“con-abundant”) codon for the nucleic acid sequence; and/or (ii) replacing a con-abundant codon in the nucleic acid sequence with a con-rare codon for the nucleic acid sequence, thereby providing a modified nucleic acid sequence which is contextually modified (“con-modified”).

In another aspect, provided herein is a method of modifying, e.g., contextually modifying, a nucleic acid sequence for a target cell or a tissue, comprising: replacing a contextually rare codon (“con-rare codon”) with a different codon, e.g., a codon that is contextually abundant (“con-abundant”); and/or replacing a con-abundant codon with a different codon, e.g., a codon that is con-rare, thereby providing a modified nucleic acid sequence which is contextually modified (“con-modified”).

In an aspect, the disclosure provides a method of modifying, e.g., contextually modifying, a nucleic acid sequence in a target cell or a tissue, comprising: (a) acquiring a value for a contextually rare codon (“con-rare codon”), wherein the value is a function of one or more of the following factors, e.g., by evaluating or determining one or more of the following factors: (1) the sequence of the codon; (2) the availability of a corresponding tRNA, e.g., charged tRNA, for that con-rare codon in a target cell or tissue, e.g., one or more iso-acceptor tRNA molecules; (3) the expression profile (or proteomic properties) of the target cell or tissue (e.g., the abundance of expression of other proteins which include the con-rare codon); (4) the proportion of the tRNAs corresponding to the con-rare codon which are charged; and (5) the iso-decoder isotype of the tRNA corresponding to the con-rare codon; (b) replacing the con-rare codon with a different codon, e.g., a codon that is contextually abundant (“con-abundant”).

In yet another aspect, provided herein is a method of identifying a nucleic acid sequence containing a contextually-rare codon (“con-rare codon”) comprising: acquiring a value for a con-rare codon in the nucleic acid sequence, wherein the value is a function of one or more of the following factors, e.g., by evaluating or determining one or more of the following factors: (1) the sequence of the codon; (2) the availability of a corresponding tRNA, e.g., charged tRNA, for that con-rare codon in a target cell or tissue, e.g., one or more iso-acceptor tRNA molecules; (3) the expression profile (or proteomic properties) of the target cell or tissue (e.g., the abundance of expression of other proteins which include the con-rare codon); (4) the proportion of the tRNAs corresponding to the con-rare codon which are charged; and (5) the iso-decoder isotype of the tRNA corresponding to the con-rare codon, thereby identifying the con-rare codon in the nucleic acid sequence.

In another aspect, the disclosure provides a method of making a contextually modified (“con-modified”) nucleic acid, e.g., a DNA or RNA sequence, sequence for a target cell or a tissue, comprising: synthesizing a nucleic acid sequence comprising a codon that differs in contextual-rareness (“con-rarity”) from the corresponding codon of a reference sequence, e.g., a parental sequence, a starting sequence, a wildtype sequence or a conventionally optimized nucleic acid sequence, thereby making a con-modified nucleic acid sequence. In an embodiment, the con-modified nucleic acid sequence comprises one less or one more con-rare codon than a reference sequence.

In an embodiment in any of the methods described herein, the target cell or tissue comprises or is associated with, or correlated (negatively or positively) with, an unwanted characteristic or a selected characteristic. In an embodiment, the target cell or tissue comprises or is associated with, or correlated (negatively or positively) with, a disease or disorder. In an embodiment, the disease or disorder comprises a cancer. In an embodiment, the target cell or tissue is characterized by unwanted proliferation, e.g., benign or malignant proliferation. In some embodiments, the target cell or tissue is a cancer cell.

In an embodiment, the disease or disorder comprises a haploinsufficiency disorder, e.g., a disease in which an allele of a gene has a loss-of-function lesion, e.g., a total loss of function lesion. Exemplary haploinsufficiency disorders include GLUT1 deficiency syndrome 1, GLUT1 deficiency syndrome 2, a disorder caused by a GATA2 mutation (e.g., GATA2 deficiency; monocyte, B and NK lymphocyte deficiency; Emberger syndrome; monocytopenia and Mycobacterium avium complex/dendritic cell), Coffin-Siris syndrome 2, Charcot-Marie-Tooth disease, Robinow syndrome, Takenouchi-Kosaki syndrome, chromosome 1p35 deletion syndrome, chromosome 2p12-p11.2 deletion syndrome, WHIM syndrome, Mowat-Wilson syndrome, and Dravet syndrome.

In an embodiment, the target cell or tissue comprises a metabolic state or condition.

In an embodiment, the target cell or tissue comprises or is associated with a genetic event, e.g., a mutation, e.g., a point mutation, a rearrangement, a translocation, an insertion, or a deletion. In an embodiment, the genetic event comprises a single nucleotide polymorphism (SNP) or other marker. In an embodiment, the genetic event is associate with, or correlated (negatively or positively) with, a disease or disorder or a predisposition to a disease or disorder. In an embodiment, the target cell or tissue comprises or is associated with, or correlated (negatively or positively) with, a pattern of gene expression, e.g., unwanted or insufficient expression of a gene.

In an embodiment, the target cell or tissue comprises or is associated with an epigenetic event, e.g., histone modification, e.g., an epigenetic event which is correlated (negatively or positively) with a disease or disorder or a predisposition to a disease or disorder.

In an embodiment, the target cell or tissue comprises a product, e.g., a nucleic acid (e.g., an RNA), protein, lipid, or sugar, associated with, or correlated (negatively or positively) with, a disorder or disease. In an embodiment, the cell or tissue produces a product, e.g., a nucleic acid (e.g., an RNA), protein, lipid, or sugar, the presence thereof is associated with, or correlated (negatively or positively) with, an unwanted state, e.g., a disease or disorder.

In an embodiment, the cell or tissue fails to produce, or fails to produce a sufficient amount of, a product, e.g., a nucleic acid (e.g., an RNA), protein, lipid, or sugar, and the absence or insufficient amount of such product is associated with, or correlated (negatively or positively) with, an unwanted state, e.g., a disease or disorder.

In an embodiment, the target cell or tissue comprises developmental stage, e.g., embryonic, fetal, immature, mature, or senescent. In an embodiment, the target cell or a cell in the target tissue comprises a stage in the cell cycle, e.g., G0, G1, S, G2, or M. In an embodiment, the target cell or tissue is non-proliferative or quiescent. In an embodiment, the target cell or tissue is proliferative. In an embodiment the cell or tissue comprises a hematopoietic cell or tissue, e.g., a fibroblast. In an embodiment the cell or tissue comprises a hepatic cell or tissue. In an embodiment the cell or tissue comprises a renal cell or tissue. In an embodiment the cell or tissue comprises a neural cell or tissue, e.g., a neuron. In an embodiment the cell or tissue comprises a muscle cell or tissue. In an embodiment the cell or tissue comprises a skin cell or tissue.

In another aspect, provided herein is a method of manufacturing a cell comprising a contextually-modified (“con-modified”) nucleic acid sequence, comprising: forming the con-modified nucleic acid sequence in the cell, e.g., by introducing the con-modified nucleic acid into the cell, thereby manufacturing a cell comprising a con-modified nucleic acid sequence. In an embodiment, the cell is homologous to the con-modified nucleic acid sequence. In an embodiment, the cell is heterologous to the con-modified nucleic acid sequence.

Also provided herein, in another aspect, is a contextually-modified (“con-modified”) nucleic acid sequence and a cell comprising contextually-modified (“con-modified”) nucleic acid sequence. In an embodiment, the con-modified nucleic acid sequence comprises DNA. In an embodiment, the con-modified nucleic acid sequence comprises an RNA, e.g., an RNA that can be translated into a polypeptide, e.g., an mRNA.

In another aspect, the disclosure provides a method of expressing a nucleic acid sequence or obtaining the product of a nucleic acid sequence, comprising: providing a contextually-modified (“con-modified”) nucleic acid sequence; contacting the con-modified nucleic sequence with a cell or a cell-free production system; and expressing the con-modified nucleic acid sequence, wherein the con-modified nucleic acid sequence comprises a codon that differs in contextual-rareness (“con-rarity”) from the corresponding codon of a reference sequence, e.g., a parental sequence, a starting sequence, a wildtype sequence or a conventionally optimized nucleic acid sequence, thereby expressing a nucleic acid sequence or obtaining the product of a nucleic acid sequence.

In yet another aspect, provided herein is a system for identifying a codon-value for a nucleic acid sequence for expression in a target cell or tissue, comprising: a computer, e.g., a general purpose computer, configured for: optionally, acquiring a nucleic acid sequence from the target cell or tissue; a) acquiring, e.g., calculating, a codon-value which is a function of the contextual-rareness (“con-rarity”) of a sequence-codon in a sequence, wherein the con-rarity of a codon is a function of one or more of the following factors, e.g., by evaluating one or more of the following factors: (1) the sequence of the codon; (2) the availability of a corresponding tRNA, e.g., charged tRNA, for that con-rare codon in a target cell or tissue, e.g., one or more isoacceptor tRNA molecules; (3) the expression profile (or proteomic properties) of the target cell or tissue (e.g., the abundance of expression of other proteins which include the con-rare codon); (4) the proportion of the tRNAs corresponding to the con-rare codon which are charged; and (5) the iso-decoder isotype of the t RNA corresponding to the con-rare codon, b) memorializing, e.g., recording, the codon-value on a medium, e.g., in a machine readable medium, thereby identifying the codon-value for the nucleic acid sequence.

The genetic code allows for 64 possible permutations, or combinations, of three-letter nucleotide sequences (i.e. codons) that can be made from the four nucleotides C, G, A and T. These 64 codons encode 20 amino acids and 3 stop signals, indicating a redundancy (or degeneracy) in the genetic code. Due to this degeneracy of the genetic code, most amino acids can be encoded by more than one trinucleotide codon. Therefore, the DNA sequence of a nucleic acid sequence, e.g., gene, encoding a protein can be modified by synonymous nucleotide substitutions (i.e. different codons encoding the same amino acid) without altering the amino acid sequence of the encoded protein.

Codon usage can differ at an organismal level. The difference in codon usage is also referred to as codon usage bias. This phenomenon is particularly relevant for heterologous protein expression since different organisms exhibit bias towards use of certain codons over others for the same amino acid. Some species are known to avoid certain codons almost entirely or use them rarely (“rare codons”). Accordingly, practitioners may “codon-optimize” a sequence in order to more efficiently express a protein derived from one source in a heterologous organism. For example, to express a non-human protein in a human host cell, tissue or subject, the protein's nucleic acid sequence is often codon optimized for expression in human cells based on known human codon bias.

The inventors have discovered that the codon identity and function is in fact highly contextual and varied within an organism. Disclosed herein, inter alia, are methods of identifying a contextually rare codon (“con-rare codon”), methods of modifying a nucleic acid sequence comprising one or a plurality of con-rare codons and uses of said methods. A con-rare codon is a codon that is limiting for a production parameter, e.g., an expression parameter or a signaling parameter, for a nucleic acid sequence, e.g., gene. In an embodiment, identification or evaluation of a con-rare codon comprises evaluating contextual rareness (con-rarity) which is a function of normalized proteome codon count and tRNA availability in a specific or selected target tissue or cell. The specific or selected target tissue or cell exists in a particular context which may be, e.g., a cell or tissue type in a particular developmental stage, a cell or tissue type in a particular disease state, a cell present in a particular extracellular milieu, a cell which has undergone a change (e.g., differentiation, proliferation or activation); a cell with finite proliferative capacity (e.g., a primary cell); a cell with unlimited proliferative capacity (e.g., an immortalized cell); a cell with differential potential (e.g., a totipotent cell, a multipotent cell or a pluripotent cell); a differentiated cell; a somatic cell; a germline cell; or a cell with preselected level of RNA or protein expression. For example, the specific or selected target tissue or cell is specific for a particular tissue, e.g., a tissue formed by a germ layer, e.g., mesoderm, ectoderm or endoderm.

In an embodiment, contextual rareness (con-rarity) is a measure of codon frequency that is contextually dependent on tRNA availability or activity levels in a specific or selected target tissue or cell. Normalized proteome codon count is a function of codon count per nucleic acid sequence and the expression profile (or proteomic properties) of a target tissue or cell. In an embodiment, a tRNA corresponding to a con-rare codon is less available in amount or activity compared to the demand of said tRNA based on the codon count per gene, and thus the codon corresponding to said tRNA may be categorized as a con-rare codon.

For example, in a specific or selected cell where (on average) codon X appears Y times for every 100 codons associated with the cells' proteome, codon X is a con-rare codon if less than 10Y, 5Y, Y, 0.5Y, 0.2Y, or 0.1Y % of the existing, functionally available, temporally available, or translationally-competent tRNAs in that same cell correspond to codon X. In an embodiment, the level is Y. As another example, in a specific or selected cell where (on average) codon X appears 3 times for every 100 codons associated with the cells' proteome, codon X is a con-rare codon if less than 3% of the existing, functionally available, temporally available, or translationally-competent tRNAs in that same cell correspond to codon X.

In an embodiment, con-rarity takes into account both the supply of tRNAs corresponding to the codon and the demand placed on that supply in the context of a specific or selected cell or tissue.

Methods described herein allow for the contextual modification of codons (in embodiments, the replacement of a con-rare codon with a codon that is not con-rare (e.g., is con-abundant); or replacement of a con-abundant codon (i.e. a codon that is not a con-rare codon) with a con-rare codon) in a nucleic acid sequence, e.g., gene, to be expressed in a cell, e.g., a heterologous nucleic acid sequence, e.g., gene, expressed in a cell. The resultant modified nucleic acid sequence is also referred to as a contextually modified nucleic acid sequence (“con-modified nucleic acid sequence”). In an embodiment, a con-modified nucleic acid sequence results in a different production parameter, e.g., an expression parameter or signaling parameter, compared to that seen with expression of a reference nucleic acid sequence. In an embodiment, a con-modified nucleic acid sequence results in an increase in a production parameter, e.g., an increase in protein production, translation (e.g., rate of translation), folding, and/or stability. In an embodiment, a con-modified nucleic acid sequence results in a decrease in a production parameter, e.g., a decrease in protein production, translation (e.g., rate of translation), folding, and/or stability.

The contextual modification of codons approach is based on adjusting codon usage in the nucleic acid sequence, e.g., gene, to the contextual availability of a tRNA in a specific or selected target cell or tissue. The approach can take into account a number of factors, including, the availability, e.g., abundance, of a tRNA corresponding to a con-rare codon in the target tissue or cell, or the demand placed on a tRNA by the codons of other expressed nucleic acid sequences, e.g., gene(s) in the target tissue or cell. E.g., codon modification of a nucleic acid sequence can take into account the expression profile (or proteomic properties) in the specific or selected target cell or tissue of other nucleic acid sequences having con-rare codons, e.g., gene, and/or the frequency or proportion of appearance of the con-rare codon in an expressed nucleic acid sequence having a con-rare codon, e.g., gene.

Additional features of any of the aforesaid methods of sequence optimization, compositions comprising a sequence optimized using a method disclosed herein and systems for optimizing a sequence include one or more of the following enumerated embodiments.

Those skilled in the art will recognize or be able to ascertain using no more than routine experimentation, many equivalents to the specific embodiments of the disclosure described herein. Such equivalents are intended to be encompassed by the following enumerated embodiments.

### Enumerated Embodiments

E1. A method of modifying, e.g., contextually modifying, a nucleic acid sequence, e.g., a nucleic acid sequence comprising an open reading frame (ORF) or coding sequence (CDS), for expression in a target cell or tissue comprising:

a) acquiring the identity of a contextually rare codon (“con-rare codon”) for the nucleic acid sequence in the target cell or tissue;

b)(i) replacing a con-rare codon in the nucleic acid sequence with a contextually abundant (“con-abundant”) codon for the nucleic acid sequence; and/or (ii) replacing a con-abundant codon in the nucleic acid sequence with a con-rare codon for the nucleic acid sequence,

thereby providing a modified nucleic acid sequence which is contextually modified (“con-modified”).

E2. The method of embodiment E1, comprising b)(i).
E3. The method of any one of embodiments E1 or E2, comprising b)(ii).
E4. The method of any one of the preceding embodiments, comprising acquiring the sequence of the nucleic acid sequence.
E5. The method of any one of the preceding embodiments, comprising transmitting the con-modified nucleic acid sequence to another entity.
E6. The method of any one of the preceding embodiments, comprising synthesizing the con-modified nucleic acid sequence.
E7. The method of any one of the preceding embodiments, comprising forming a bond between a first nucleotide and a second nucleotide to provide the con-modified nucleic acid sequence.
E8. A method of modifying, e.g., contextually modifying, a nucleic acid sequence, e.g., a nucleic acid sequence comprising an open reading frame (ORF) or coding sequence (CDS), for a target cell or a tissue, comprising:

replacing a contextually rare codon (“con-rare codon”) with a different codon, e.g., a codon that is contextually abundant (“con-abundant”); and/or

replacing a con-abundant codon with a different codon, e.g., a codon that is con-rare,

thereby providing a modified nucleic acid sequence which is contextually modified (“con-modified”).

E9. The method of embodiment E8, comprising synthesizing the con-modified nucleic acid sequence.
E10. The method of embodiment E7 or E8, comprising forming a bond between a first nucleotide and a second nucleotide to provide the con-modified nucleic acid sequence.
E11. A method of modifying, e.g., contextually modifying, a nucleic acid sequence, e.g., a nucleic acid sequence comprising an open reading frame (ORF) or coding sequence (CDS), in a target cell or a tissue, comprising:

(a) acquiring a value for a contextually rare codon (“con-rare codon”), wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon,

(b) replacing the con-rare codon with a different codon, e.g., a codon that is contextually abundant (“con-abundant”).

E12. A method of identifying a contextually rare codon (“con-rare codon”) comprising,

acquiring a value for a con-rare codon, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon,

thereby identifying the con-rare codon.

E13. A method of identifying a nucleic acid sequence having a contextually rare codon (“con-rare codon”) comprising,

acquiring a value for a con-rare codon in the nucleic acid sequence, wherein the value is a function of one or more of the following factors, e.g., by evaluating or determining one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon, thereby identifying the con-rare codon in the nucleic acid sequence.

E14. A method of making a contextually modified (“con-modified”) nucleic acid sequence, e.g., a DNA or RNA sequence, for a target cell or a tissue, comprising:

synthesizing a nucleic acid sequence comprising a codon that differs in contextual rareness (“con-rarity”) from the corresponding codon of a reference sequence, e.g., a parental sequence, a starting sequence, a wildtype sequence or a conventionally optimized nucleic acid sequence,

thereby making a con-modified nucleic acid sequence.

E15. The method of embodiment E14, wherein the con-modified nucleic acid sequence comprises one less or one more con-rare codon than a reference sequence.
E16. The method of embodiment E14 or E15, which comprises replacing at least 1, 2, 3, 4, 5, 10, 15, 20, 30, 50, 100 or all of the con-rare codons with a contextually abundant (“con-abundant”) codon in the target cell or tissue.
E17. The method of embodiment E14 or E15, which comprises replacing about 1 to 100, 2 to 100, 3 to 100, 4 to 100, 5 to 100, 10 to 100, 15 to 100, 20 to 100, 30 to 100, 50 to 100, 1 to 50, 1 to 30, 1 to 20, 1 to 15, 1 to 10, 1 to 5, 1 to 4, 1 to 3, or 1 to 2 of the con-rare codons with a con-abundant codon in the target cell or tissue.
E18. The method of embodiment E14 or E15, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of the con-rare codons with a con-abundant codon in the target cell or tissue.
E19. The method of embodiment E14 or E15, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of the con-rare codons with a con-abundant codon in the target cell or tissue.
E20. The method of embodiment E14 or E15, which comprises replacing at least 1, 2, 3, 4, 5, 10, 15, 20, 30, 50, 100 or all of a con-abundant codon with a con-rare codon in the target cell or tissue.
E21. The method of embodiment E14 or E15, which comprises replacing about 1 to 100, 2 to 100, 3 to 100, 4 to 100, 5 to 100, 10 to 100, 15 to 100, 20 to 100, 30 to 100, 50 to 100, 1 to 50, 1 to 30, 1 to 20, 1 to 15, 1 to 10, 1 to 5, 1 to 4, 1 to 3, or 1 to 2 of a con-abundant codon with a con-rare codon in the target cell or tissue.
E22. The method of embodiment E14 or E15, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of a con-abundant codon with a con-rare codon in the target cell or tissue.
E23. The method of embodiment E14 or E15, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of a con-abundant codon with a con-rare codon in the target cell or tissue.
E24. The method of any of the preceding embodiments, wherein the target cell or tissue is a specific or selected target cell or tissue, e.g., a cell or tissue type in a particular developmental stage; a cell or tissue type in a particular disease state; or a cell present in a particular extracellular milieu.
E25. The method of any one of the preceding embodiments, wherein the target cell or tissue comprises or is associated, or correlated (negatively or positively) with, an unwanted characteristic or a selected characteristic.
E26. The method of any one of the preceding embodiments, wherein the target cell or tissue comprises or is associated, or correlated (negatively or positively) with, a disease or disorder.
E27. The method of embodiment of E26, wherein the disease or disorder comprises a cancer or a haploinsufficiency disorder.
E28. The method of any one of the preceding embodiments, wherein the target cell or tissue is characterized by unwanted proliferation, e.g., benign or malignant proliferation.
E29. The method of any one of the preceding embodiments, wherein the target cell or tissue comprises or is associated with a genetic event, e.g., a mutation, e.g., a point mutation, a rearrangement, a translocation, an insertion, or a deletion.
E30. The method of any one of preceding embodiments, wherein the target cell or tissue comprises or is associated with an epigenetic event, e.g., histone modification, e.g., an epigenetic event which is correlated (negatively or positively) with a disease or disorder or a predisposition to a disease or disorder.
E31. The method of any one of the preceding embodiments, wherein the target cell or tissue comprises a product, e.g., a nucleic acid (e.g., an RNA), protein, lipid, sugar, associated with, or correlated (negatively or positively) with, a disorder or disease.
E32. The method of embodiments of E30 or E31, wherein the disease or disorder comprises a cancer or a haploinsufficiency disorder.
E33. A contextually-modified (“con-modified”) nucleic acid sequence.
E34. The con-modified nucleic acid sequence of embodiment E33, wherein the con-modified nucleic acid sequence comprises:

(i) a DNA; or

(ii) an RNA, e.g., an RNA that can be translated into a polypeptide, e.g., an mRNA.

E35. The con-modified of nucleic acid sequence of embodiment E33 or E34, wherein the con-modified nucleic acid sequence is made according to the method of any one of embodiments E1-E32.
E36. The con-modified nucleic acid sequence of any one of embodiments E33-E35, which is provided to, e.g., introduced into, a cell (e.g., a host cell or target cell) or tissue.
E37. A composition comprising a preparation of RNA which encodes a polypeptide product, wherein the RNA comprises a codon that differs in contextual rareness (“con-rarity”) from the corresponding codon of a reference sequence, e.g., a parental sequence, a starting sequence, a wildtype sequence or a conventionally optimized nucleic acid sequence.
E38. The composition of embodiment E37, wherein the RNA comprises one less or one more con-rare codons than a reference sequence.
E39. The RNA preparation of embodiment E37, wherein the RNA comprises one less or one more con-abundant codons than a reference sequence.
E40. The RNA preparation of any one of embodiments E37-E39, which comprises 2, 3, 4, 5, 10, 20, or 30 less con-rare codons than the reference sequence.
E41. The RNA preparation of any one of embodiments E37-E39, which comprises 2, 3, 4, 5, 10, 20, or 30 more con-rare codons than the reference sequence.
E42. The RNA preparation of any one of embodiments E37-E41, which comprises 2, 3, 4, 5, 10, 20, or 30 less con-abundant codons than the reference sequence.
E43. The RNA preparation of any one of embodiments E37-E41, which comprises 2, 3, 4, 5, 10, 20, or 30 more con-abundant codons than the reference sequence.
E44. The RNA preparation of any one of embodiments E37-E43, wherein the preparation is a pharmaceutical preparation.
E45. The RNA preparation of any one of embodiments E37-E43, wherein the RNA comprises a modification which prevents, e.g., inhibits, the degradation of the RNA, or a modification that alters RNA localization.
E46. The RNA preparation of embodiment E45, wherein the modification comprises one or more atoms of a pyrimidine nucleobase replaced or substituted with amino or thiol or a different substitution, e.g., as described in WO 2014/152027 A1.
E47. The RNA preparation of any one of embodiments E37-E46, wherein the RNA comprises a 5′UTR, a 3′ UTR, a poly A tail, and/or a binding site for a micro RNA.
E48. The RNA preparation of any one of embodiments E37-E47, wherein the RNA comprises a targeting moiety which targets the RNA to a target tissue or cell.
E49. The RNA preparation of any one of embodiments E37-E48, wherein the RNA is disposed in a delivery entity, e.g., a nanoparticle, liposome, or a delivery entity described herein.
E50. A method of manufacturing a cell comprising a contextually-modified (“con-modified”) nucleic acid sequence, comprising:

forming the con-modified nucleic acid sequence in the cell, e.g., by introducing the con-modified nucleic acid into the cell,

thereby manufacturing a cell comprising a con-modified nucleic acid sequence.

E51. The method of embodiment E50, wherein the cell is homologous to the con-modified nucleic acid sequence.
E52. The method of embodiment E50, wherein the cell is heterologous to the con-modified nucleic acid sequence.
E53. The method of any one of embodiments E50-E52, further comprising allowing the cell comprising the con-modified nucleic acid sequence to divide to form a population of cells comprising the con-modified nucleic acid sequence.
E54. The method of any one of embodiments E50-E53, further comprising culturing the cell under conditions that allow for the expression of a product, e.g., RNA or polypeptide, from the con-modified nucleic acid sequence.
E55. The method of any one of embodiments E50-E54, wherein the con-modified nucleic acid sequence comprises DNA.
E56. The method of any one of embodiments E50-E55, wherein the con-modified nucleic acid sequence comprises RNA, e.g., mRNA or another RNA having an open reading frame, or which is translated.
E57. The method of any one of embodiments E50-E56, comprising expressing a product, e.g., a polypeptide or RNA, from the con-modified nucleic acid sequence.
E58. The method of any one of embodiments E50-E57, comprising

providing a con-modified nucleic acid sequence;

optionally, contacting the cell with the con-modified nucleic acid sequence; and

maintaining the cell under conditions that allow for expression of a product of the con-modified nucleic acid sequence,

wherein the con-modified nucleic acid sequence comprises a codon that differs in con-rarity from the corresponding codon of a reference sequence, e.g., a parental sequence, a starting sequence, a wildtype sequence or a conventionally optimized nucleic acid sequence.

E59. The method of any one of embodiments E50-E58, wherein the con-modified nucleic acid sequence comprises one less or one more con-rare codons than a reference sequence.
E60. The method of any one of embodiments E50-E59, wherein the con-modified nucleic acid sequence comprises one less or one more con-abundant codons than a reference sequence.
E61. The method of embodiment E58 or E59, which comprises 2, 3, 4, 5, 10, 20, or 30 less con-rare codons than the reference sequence.
E62. The method of embodiment E58 or E59, which comprises 2, 3, 4, 5, 10, 20, or 30 more con-rare codons than the reference sequence.
E63. The method of embodiment E58 or E60, which comprises 2, 3, 4, 5, 10, 20, or 30 less con-abundant codons than the reference sequence.
E64. The method of embodiment E58 or E60, which comprises 2, 3, 4, 5, 10, 20, or 30 more con-abundant codons than the reference sequence.
E65. The method of any one of embodiments E50-E64, wherein the cell is a host cell.
E66. The method of embodiment E65, wherein the cell is a mammalian cell, e.g., a human cell, a murine cell, or a rodent cell.
E67. The method of embodiment E65, wherein the cell is a non-mammalian cell, e.g., a bacterial cell, an insect cell or a yeast cell.
E68. The method of any one of embodiments E65-E67, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
E69. The method of any one of embodiments E50-E68, wherein the cell is a target cell.
E70. The method of embodiment E69, wherein the target cell is a cell that is, or has been identified as having a con-rare codon.
E71. The method of any one of embodiments E50-E70, wherein the cell expresses a product, e.g., a RNA or polypeptide, of the con-optimized nucleic acid sequence.
E72. The method of any one of embodiments E50-E71, wherein the cell expresses at least 105%, 110%, 115%, 120%, 130%, 140%, 150%, 200%, 300%, 400%, 500%, 1000%, 2000%, 3000%, 4000%, or 5000% more of the product (e.g., polypeptide of the con-optimized nucleic acid sequence) compared to a reference value, e.g., the level of the product expressed from a reference cell maintained under similar conditions.
E73. The method of embodiment E72, wherein the reference cell comprises a wildtype nucleic acid sequence or a conventionally optimized nucleic acid sequence.
E74. The method of any one of embodiments E50-E73, comprising introducing the con-modified nucleic acid sequence into a cell (e.g., a host cell or target cell) or tissue.
E75. The method of any one of embodiments E50-E74, wherein the con-modified nucleic acid sequence results in a product, e.g., a polypeptide encoded by the con-modified nucleic acid sequence, or an RNA (e.g., messenger RNA (mRNA) which can be translated into a polypeptide).
E76. The method of any one of embodiments E50-E75, comprising recovering (e.g., purifying) a product made by the con-modified nucleic acid sequence, e.g., a polypeptide or RNA.
E77. The method of any one of embodiments E50-E76, wherein the con-modified nucleic acid sequence results in a product (e.g., polypeptide encoded by the con-modified nucleic acid sequence) having a production parameter that is different from a production parameter of a product of the reference nucleic acid sequence.
E78. The method of embodiment E77, wherein the production parameter is an expression parameter (e.g., as described herein) or a signaling parameter (e.g., as described herein).
E79. The method of any one of embodiments E77-E78, wherein the con-modified nucleic acid sequence results in a product (e.g., polypeptide encoded by the con-modified nucleic acid sequence) which is produced, e.g., expressed, at least 105%, 110%, 115%, 120%, 130%, 140%, 150%, 200%, 300%, 400%, 500%, 1000%, 2000%, 3000%, 4000%, or 5000% more compared to the production, e.g., expression, of a similar product (e.g., polypeptide) produced, e.g., expressed, by a reference sequence, e.g., a non-modified nucleic acid sequence (e.g., wildtype or a conventionally optimized nucleic acid sequence).
E80. The method of any one of embodiments E77-E78, wherein the product of the con-modified nucleic acid sequence is produced (e.g., expressed), about 105% to 5000%, 105% to 4000%, 105% to 3000%, 105% to 2000%, 105% to 1000%, 105% to 500%, 105% to 400%, 105%, to 300%, 105% to 250%, 105% to 200%, 105% to 150%, 105% to 140%, 105% to 130%, 105% to 125%, 105% to 120%, 105% to 115%, 105% to 110%, 110% to 5000%, 115% to 5000%, 120% to 5000%, 125% to 5000%, 130% to 5000%, 140% to 5000%, 150% to 5000%, 200% to 5000%, 300% to 5000%, 400% to 5000%, 500% to 5000%, 1000% to 5000%, 2000% to 5000%, 3000% to 5000%, or 4000% to 5000%, more compared to the production, e.g., expression, of a similar product (e.g., polypeptide) produced, e.g., expressed, by a reference sequence, e.g., a non-modified nucleic acid sequence (e.g., wildtype or a conventionally optimized nucleic acid sequence).
E81. The method of any one of embodiments E77-E80, wherein the con-modified nucleic acid sequence results in a product (e.g., polypeptide encoded by the con-modified nucleic acid sequence) which is produced, e.g., expressed, at least 105%, 110%, 115%, 120%, 130%, 140%, 150%, 200%, 300%, 400%, 500%, 1000%, 2000%, 3000%, 4000%, or 5000% lesser compared to the production, e.g., expression, of a similar product (e.g., polypeptide) produced, e.g., expressed, by a reference sequence, e.g., a non-modified nucleic acid sequence (e.g., wildtype or a conventionally optimized nucleic acid sequence).
E82. The method of any one of embodiments E77-E80, wherein the product of the con-modified nucleic acid sequence is produced (e.g., expressed), about 105% to 5000%, 105% to 4000%, 105% to 3000%, 105% to 2000%, 105% to 1000%, 105% to 500%, 105% to 400%, 105%, to 300%, 105% to 250%, 105% to 200%, 105% to 150%, 105% to 140%, 105% to 130%, 105% to 125%, 105% to 120%, 105% to 115%, 105% to 110%, 110% to 5000%, 115% to 5000%, 120% to 5000%, 125% to 5000%, 130% to 5000%, 140% to 5000%, 150% to 5000%, 200% to 5000%, 300% to 5000%, 400% to 5000%, 500% to 5000%, 1000% to 5000%, 2000% to 5000%, 3000% to 5000%, or 4000% to 5000%, lesser compared to the production, e.g., expression, of a similar product (e.g., polypeptide) produced, e.g., expressed, by a reference sequence, e.g., a non-modified nucleic acid sequence (e.g., wildtype or a conventionally optimized nucleic acid sequence).
E83. A cell comprising a contextually-modified (“con-modified”) nucleic acid sequence.
E84. A preparation of cells comprising a contextually-modified (“con-modified”) nucleic acid sequence.
E85. The cell or preparation of cells of embodiment E83-E84, wherein the cell or preparation of cells comprises a con-modified nucleic acid sequence which comprises DNA.
E86. The cell or preparation of cells of embodiment E83-E84, wherein the cell or preparation of cells comprises a con-modified nucleic acid sequence which comprises RNA.
E87. The cell or preparation of cells of any one of embodiments E83-E86, wherein the cell or preparation of cells comprises a product of the con-modified nucleic acid sequence, e.g., wherein the product comprises an RNA or a polypeptide.
E88. The cell or preparation of cells of any one of embodiments E83-E87, wherein the con-modified nucleic acid is made according to the method of any one of embodiment E1-E49.
E89. The cell or preparation of cells of any one of embodiments E83-E87, wherein the cell or preparation of cells is contacted with the con-modified nucleic acid sequence.
E90. The cell or preparation of cells of any one of embodiments E83-E89, wherein the cell or preparation of cells is cultured under conditions that allow for expression of a product, e.g., polypeptide or RNA, of the con-modified nucleic acid sequence.
E91. The cell or preparation of cells of any one of embodiments E83-E90, comprising recovering (e.g., purifying) a product made by the con-modified nucleic acid sequence, e.g., a polypeptide or RNA.
E92. The cell or preparation of cells of any one of embodiments E83-E91, wherein the con-modified nucleic acid sequence results in a product (e.g., polypeptide encoded by the con-modified nucleic acid sequence) having a production parameter that is different from a production parameter of a product of the reference nucleic acid sequence.
E93. The cell or preparation of cells of embodiment E92, wherein the production parameter is an expression parameter (e.g., as described herein) or a signaling parameter (e.g., as described herein).
E94. The cell or preparation of cells of embodiment E92 or E93, wherein the con-modified nucleic acid sequence results in a product (e.g., polypeptide encoded by the con-modified nucleic acid sequence) which is produced, e.g., expressed, at least 105%, 110%, 115%, 120%, 130%, 140%, 150%, 200%, 300%, 400%, 500%, 1000%, 2000%, 3000%, 4000%, or 5000% more compared to the production, e.g., expression, of a similar product (e.g., polypeptide) produced, e.g., expressed, by a cell comprising a reference sequence, e.g., a non-modified nucleic acid sequence (e.g., wildtype or a conventionally optimized nucleic acid sequence).
E95. The cell or preparation of cells of any one of embodiments E92-E94, wherein the product of the con-modified nucleic acid sequence is produced (e.g., expressed), about 105% to 5000%, 105% to 4000%, 105% to 3000%, 105% to 2000%, 105% to 1000%, 105% to 500%, 105% to 400%, 105%, to 300%, 105% to 250%, 105% to 200%, 105% to 150%, 105% to 140%, 105% to 130%, 105% to 125%, 105% to 120%, 105% to 115%, 105% to 110%, 110% to 5000%, 115% to 5000%, 120% to 5000%, 125% to 5000%, 130% to 5000%, 140% to 5000%, 150% to 5000%, 200% to 5000%, 300% to 5000%, 400% to 5000%, 500% to 5000%, 1000% to 5000%, 2000% to 5000%, 3000% to 5000%, or 4000% to 5000%, more compared to the production, e.g., expression, of a similar product (e.g., polypeptide) produced, e.g., expressed, by a cell comprising a reference sequence, e.g., a non-modified nucleic acid sequence (e.g., wildtype or a conventionally optimized nucleic acid sequence).
E96. The cell or preparation of cells of any one of embodiments E92-E95, wherein the con-modified nucleic acid sequence results in a product (e.g., polypeptide encoded by the con-modified nucleic acid sequence) which is produced, e.g., expressed, at least 105%, 110%, 115%, 120%, 130%, 140%, 150%, 200%, 300%, 400%, 500%, 1000%, 2000%, 3000%, 4000%, or 5000% lesser compared to the production, e.g., expression, of a similar product (e.g., polypeptide) produced, e.g., expressed, by a cell comprising a reference sequence, e.g., a non-modified nucleic acid sequence (e.g., wildtype or a conventionally optimized nucleic acid sequence).
E97. The cell or preparation of cells of any one of embodiments E92-E96, wherein the product of the con-modified nucleic acid sequence is produced (e.g., expressed), about 105% to 5000%, 105% to 4000%, 105% to 3000%, 105% to 2000%, 105% to 1000%, 105% to 500%, 105% to 400%, 105%, to 300%, 105% to 250%, 105% to 200%, 105% to 150%, 105% to 140%, 105% to 130%, 105% to 125%, 105% to 120%, 105% to 115%, 105% to 110%, 110% to 5000%, 115% to 5000%, 120% to 5000%, 125% to 5000%, 130% to 5000%, 140% to 5000%, 150% to 5000%, 200% to 5000%, 300% to 5000%, 400% to 5000%, 500% to 5000%, 1000% to 5000%, 2000% to 5000%, 3000% to 5000%, or 4000% to 5000%, lesser compared to the production, e.g., expression, of a similar product (e.g., polypeptide) produced, e.g., expressed, by a cell comprising a reference sequence, e.g., a non-modified nucleic acid sequence (e.g., wildtype or a conventionally optimized nucleic acid sequence).
E98. The cell or preparation of cells of any one of embodiments E83-E97, wherein the reference cell comprises an otherwise similar cell, cultured under similar conditions which:

(a) does not comprise the con-modified nucleic acid sequence;

(b) has not been contacted with the con-modified nucleic acid sequence; or

(c) comprises a nucleic acid sequence which has not been con-modified, e.g., a wildtype or a conventionally optimized nucleic acid sequence.

E99. The cell or preparation of cells of any one of embodiments E83-E98, wherein the cell is a target cell or a host cell.
E100. The cell or preparation of cells of any one of embodiments E83-E99, wherein the cell is a mammalian cell, e.g., a human cell, a murine cell, or a rodent cell.
E101. The cell or preparation of cells of any one of embodiments E83-E100, wherein the cell is a non-mammalian cell, e.g., a bacterial cell, an insect cell or a yeast cell.
E102. The cell or preparation of cells of any one of embodiments E83-E101, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
E103. The cell or preparation of cells of any one of embodiments E83-E102, wherein the cell is a target cell.
E104. The cell or preparation of cells of embodiment E103, wherein the target cell is a cell that is, or has been identified as having a con-rare codon.
E105. The cell or preparation of cells of embodiment E103 or E104, wherein for at least one con-rare codon, a tRNA corresponding to the con-rare codon is not provided to the target cell or tissue.
E106. The cell or preparation of cells of any one of embodiments E103-E105, wherein providing a tRNA corresponding to the con-rare codon does not increase expression of a product of the con-modified nucleic acid sequence.
E107. The cell or preparation of cells any one of embodiments E103-E106, wherein the target cell comprises a cell associated with a disease or disorder, e.g., a disease or disorder associated with a con-rare codon.
E108. The cell or preparation of cells of embodiment E107, wherein the cell is obtained from a subject having the disease or disorder.
E109. The cell or preparation of cells any one of embodiments E103-E108, wherein the cell is a cancer cell, e.g., a solid tumor cell (e.g., a breast cancer cell (e.g., a MCF7 cell), a pancreatic cell line (e.g. a MIA PaCa-2 cell), a lung cancer cell, or a prostate cancer cell, or a hematological cancer cell).
E110. A method of expressing a nucleic acid sequence or obtaining the product of a nucleic acid sequence, comprising:

providing a contextually-modified (“con-modified”) nucleic acid sequence;

contacting the con-modified nucleic sequence with a cell or a cell-free production system; and

expressing the con-modified nucleic acid sequence,

wherein the con-modified nucleic acid sequence comprises a codon that differs in contextual rareness (“con-rarity”) from the corresponding codon of a reference sequence, e.g., a parental sequence, a starting sequence, a wildtype sequence or a conventionally optimized nucleic acid sequence,

thereby expressing a nucleic acid sequence or obtaining the product of a nucleic acid sequence.

E111. The method of embodiment E110, wherein the con-modified nucleic acid sequence comprises at least one lesser or one more con-rare codon than the reference sequence.
E112. The method of embodiment E110 or E111, wherein the con-modified nucleic acid sequence comprises at least one lesser or one more con-abundant codons than the reference sequence.
E113. The method of embodiment E110 or E111, wherein the con-modified nucleic acid sequence comprises DNA or RNA.
E114. The method of any one of embodiments E110-E113, wherein expression of the con-modified nucleic acid sequence results in a product, e.g., an RNA or polypeptide.
E115. The method of embodiment E114, wherein the product of the con-modified nucleic acid sequence comprises a polypeptide.
E116. The method of embodiment E114, wherein the product of the con-modified nucleic acid sequence comprises RNA.
E117. The method of any one of embodiments E110-E116, wherein the con-modified nucleic acid is disposed in a cell, e.g., a host cell or a target cell.
E118. The method of embodiment E117, wherein the cell is a mammalian cell, e.g., a human cell, a murine cell, or a rodent cell.
E119. The method of embodiment E117, wherein the cell is a non-mammalian cell, e.g., a bacterial cell, an insect cell or a yeast cell.
E120. The method of embodiment E117, wherein the cell is a host cell chosen from: a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell.
E121. The method of embodiment E117, wherein the cell is a target cell.
E122. The method of embodiment E121, wherein the target cell is a cell that is, or has been identified as having a con-rare codon.
E123. The method of any one of embodiments E110-E122, wherein the con-modified nucleic acid sequence is expressed in vitro or in vivo.
E124. The method of any one of embodiments E110-E123, comprising recovering, e.g., purifying, an expressed product, e.g., a polypeptide, from the con-modified nucleic acid.
E125. The method of any one of embodiments E123 or E124, wherein the con-modified nucleic acid sequence comprises:

2, 3, 4, 5, 10, 20, or 30 less con-rare codons than the reference sequence;

2, 3, 4, 5, 10, 20, or 30 more con-rare codons than the reference sequence;

2, 3, 4, 5, 10, 20, or 30 less con-abundant codons than the reference sequence; and/or

2, 3, 4, 5, 10, 20, or 30 more con-abundant codons than the reference sequence.

E126. The method of any one of embodiments E110-E125, wherein the con-modified nucleic acid sequence results in a product (e.g., polypeptide encoded by the con-modified nucleic acid sequence) having a production parameter that is different from a production parameter of a product of the reference nucleic acid sequence.
E127. The method of embodiment E126, wherein the production parameter is an expression parameter (e.g., as described herein) or a signaling parameter (e.g., as described herein).
E128. The method of embodiment E126 or E127, resulting in at least 105%, 110%, 115%, 120%, 130%, 140%, 150%, 200%, 300%, 400%, 500%, 1000%, 2000%, 3000%, 4000%, or 5000% more expression of the product as compared with expression of a similar product from a cell or cell-free production system comprising reference sequence.
E129. The method of embodiment E126 or E127, resulting in at least 105%, 110%, 115%, 120%, 130%, 140%, 150%, 200%, 300%, 400%, 500%, 1000%, 2000%, 3000%, 4000%, or 5000% lesser expression of the product as compared with expression of a similar product from a cell or cell-free production system comprising reference sequence.
E130. The method of embodiment E128 or E129, wherein the reference sequence is a parental sequence, a starting sequence, a wildtype nucleic acid sequence or a conventionally optimized nucleic acid sequence.
E131. A method of manufacturing a product, e.g., a polypeptide or RNA, from a contextually-modified (“con-modified”) nucleic acid sequence, comprising:

providing a con-modified nucleic acid sequence under conditions that allow for transcribing the con-modified nucleic acid sequence; and

transcribing the con-modified nucleic acid sequence,

wherein the con-modified nucleic acid sequence comprises a codon that differs in contextual rareness (“con-rarity”) from the corresponding codon of a reference sequence, e.g., a parental sequence, a starting sequence, a wildtype sequence or a conventionally optimized nucleic acid sequence,

thereby manufacturing a product form the con-modified nucleic acid sequence.

E132. The method of embodiment E131, wherein the con-modified nucleic acid sequence comprises at least one lesser or one more con-rare codon than the reference sequence.
E133. The method of embodiment E131, wherein the con-modified nucleic acid sequence comprises at least one lesser or one more con-abundant codon than the reference sequence.
E134. The method of any one of embodiments E131-E133, wherein the con-modified nucleic acid sequence comprises:

2, 3, 4, 5, 10, 20, or 30 less con-rare codons than the reference sequence;

2, 3, 4, 5, 10, 20, or 30 more con-rare codons than the reference sequence;

2, 3, 4, 5, 10, 20, or 30 less con-abundant codons than the reference sequence; and/or

2, 3, 4, 5, 10, 20, or 30 more con-abundant codons than the reference sequence.

E135. The method of any one of embodiments E131-E134, wherein the con-modified nucleic acid sequence is transcribed in vitro or in vivo.
E136. The method of any one of embodiments E131-E135, comprising recovering, e.g., purifying, a product of con-modified nucleic acid sequence.
E137. The method of any one of embodiments E131-E136, wherein the con-modified nucleic acid sequence is made according to a method of any one of claims E1-E49.
E138. The method of any one of embodiments E131-E137, comprising contacting the con-modified nucleic acid sequence with a cell, e.g., a target cell or a host cell, e.g., as described herein.
E139. The method of any one of embodiments E131-E138, wherein the con-modified nucleic acid sequence results in a product (e.g., polypeptide encoded by the con-modified nucleic acid sequence) having a production parameter that is different from a production parameter of a product of the reference nucleic acid sequence.
E140. The method of embodiment E139, wherein the production parameter is an expression parameter (e.g., as described herein) or a signaling parameter (e.g., as described herein).
E141. The method of embodiment E139 or E140, resulting in at least 105%, 110%, 115%, 120%, 130%, 140%, 150%, 200%, 300%, 400%, 500%, 1000%, 2000%, 3000%, 4000%, or 5000% more product (e.g., expression of polypeptide encoded by the con-modified nucleic acid sequence) compared to the level of expression of a similar product (e.g., polypeptide) produced, e.g., expressed, by a reference sequence.
E142. The method of embodiment E139 or E140, resulting in at least 105%, 110%, 115%, 120%, 130%, 140%, 150%, 200%, 300%, 400%, 500%, 1000%, 2000%, 3000%, 4000%, or 5000% lesser product (e.g., expression of polypeptide encoded by the con-modified nucleic acid sequence) compared to the level of expression of a similar product (e.g., polypeptide) produced, e.g., expressed, by a reference sequence.
E143. The method of embodiment E141 or E142, wherein the reference sequence is a parental sequence, a starting sequence, a wildtype nucleic acid sequence or a conventionally optimized nucleic acid sequence.
E144. A method of modifying a subject sequence, e.g., a genomic sequence, comprising contacting a contextually-modified (“con-modified”) nucleic acid sequence with the subject nucleic acid sequence, e.g., a genomic sequence, under conditions which allow for incorporation of the sequence of the con-modified nucleic acid sequence into the subject nucleic acid sequence.
E145. The method of embodiment E144, wherein the incorporation is CRISPR mediated.
E146. The method of embodiment E144 or E145, wherein the incorporation is Zinc-finger mediated.
E147. The method of any one of embodiments E144-E146, wherein the incorporation is TALEN mediated.
E148. The method of any one of embodiments E144-E147, wherein the incorporation is mediated by an enzyme endogenous to a cell in which the subject nucleic acid is disposed.
E149. The method of any one of embodiments E144-E148, wherein the incorporation is mediated by an enzyme exogenous to a cell in which the subject nucleic acid is disposed.
E150. The method of any one of embodiments E144-E149, wherein the incorporation is in vivo.
E151. The method of any one of embodiments E144-E150, wherein the incorporation is in vitro.
E152. A composition comprising a contextually-modified (“con-modified”) nucleic acid sequence and a subject nucleic acid.
E153. A composition comprising a contextually-modified (“con-modified”) nucleic acid sequence and a moiety, e.g., a polypeptide, that mediates incorporation of the sequence of the con-modified nucleic acid sequence into the subject nucleic acid.
E154. The composition of embodiment E152 or E153, wherein the moiety comprises a polypeptide that mediates gene editing.
E155. The composition of any one of embodiments E153-E154, wherein the moiety comprises a CRISPR component, e.g., CAS9 or a guide RNA.
E156. A method of delivering to a subject in need thereof, a contextually-modified (“con-modified”) nucleic acid sequence, comprising administering to the subject a preparation of a con-modified nucleic acid sequence.
E157. The method of embodiment E156, comprising treating a disease or disorder in a subject by administration of an effective amount of the preparation of the con-modified nucleic acid sequence.
E158. A method of treating, or ameliorating a symptom of, a disease or disorder associated with a contextually rare codon (“con-rare codon”), comprising administering to a subject in need thereof a preparation of a contextually-modified (“con-modified”) nucleic acid sequence.
E159. The method of embodiment E158, wherein the con-modified nucleic acid sequence comprises a codon that differs in contextual rareness (“con-rarity”) from the corresponding codon of a reference sequence, e.g., a parental sequence, a starting sequence, a wildtype sequence or a conventionally optimized nucleic acid sequence.
E160. The method of embodiment E159, wherein the con-modified nucleic acid sequence comprises at least one lesser or one more con-rare codon than the reference sequence.
E161. The method of embodiment E159, wherein the con-modified nucleic acid sequence comprises at least one lesser or one more con-abundant codon than the reference sequence.
E162. The method of embodiment E160 or E161, wherein the con-modified nucleic acid sequence comprises:

2, 3, 4, 5, 10, 20, or 30 less con-rare codons than the reference sequence;

2, 3, 4, 5, 10, 20, or 30 more con-rare codons than the reference sequence;

2, 3, 4, 5, 10, 20, or 30 less con-abundant codons than the reference sequence; and/or

2, 3, 4, 5, 10, 20, or 30 more con-abundant codons than the reference sequence.

E163. The method of any one of embodiment E158-E162, wherein the disease or disorder is associated with a con-rare codon.
E164. The method of any one of embodiment E158-E163, wherein the con-modified nucleic acid sequence corresponds to a nucleic acid sequence, e.g., a gene, having the con-rare codon.
E165. The method of any one of embodiment E158-E164, wherein the preparation is a pharmaceutical preparation.
E166. The method of any one of embodiment E158-E165, wherein the con-modified nucleic acid sequence expresses a product (e.g., polypeptide or RNA).
E167. The method of any one of embodiments E158-E166, wherein the con-modified nucleic acid sequence results in a product (e.g., polypeptide encoded by the con-modified nucleic acid sequence) having a production parameter that is different from a production parameter of a product of the reference nucleic acid sequence.
E168. The method of embodiment E167, wherein the production parameter is an expression parameter (e.g., as described herein) or a signaling parameter (e.g., as described herein).
E169. The method of embodiment E167 or E168, wherein the polypeptide encoded by the con-modified nucleic acid sequence is produced, e.g., expressed, at least 105%, 110%, 115%, 120%, 130%, 140%, 150%, 200%, 300%, 400%, 500%, 1000%, 2000%, 3000%, 4000%, or 5000% more compared to the production, e.g., expression, of a similar polypeptide encoded by a reference sequence, e.g., a parental sequence, a starting sequence, a wildtype sequence or a conventionally optimized sequence.
E170. The method of embodiment E167 or E168, wherein the polypeptide encoded by the con-modified nucleic acid sequence is produced, e.g., expressed, at least 105%, 110%, 115%, 120%, 130%, 140%, 150%, 200%, 300%, 400%, 500%, 1000%, 2000%, 3000%, 4000%, or 5000% lesser compared to the production, e.g., expression, of a similar polypeptide encoded by a reference sequence, e.g., a parental sequence, a starting sequence, a wildtype sequence or a conventionally optimized sequence.
E171. A method of delivering to a subject in need thereof, a product (e.g., polypeptide or RNA) of a contextually-modified (“con-modified”) nucleic acid sequence, comprising:

providing, e.g., administering, to the subject a product (e.g., polypeptide or RNA) of a con-modified nucleic acid sequence.

E172. The method of embodiment E171, wherein the con-modified nucleic acid sequence comprises a codon that differs in contextual rareness (“con-rarity”) from the corresponding codon of a reference sequence, e.g., a parental sequence, a starting sequence, a wildtype sequence or a conventionally optimized nucleic acid sequence.
E173. The method of embodiment E172, wherein the con-modified nucleic acid sequence comprises at least one lesser or one more con-rare codon than the reference sequence.
E174. The method of embodiment E172, wherein the con-modified nucleic acid sequence comprises at least one lesser or one more con-abundant codon than the reference sequence.
E175. The method of embodiment E173 or E174, wherein the con-modified nucleic acid sequence comprises:

2, 3, 4, 5, 10, 20, or 30 less con-rare codons than the reference sequence;

2, 3, 4, 5, 10, 20, or 30 more con-rare codons than the reference sequence;

2, 3, 4, 5, 10, 20, or 30 less con-abundant codons than the reference sequence; and/or

2, 3, 4, 5, 10, 20, or 30 more con-abundant codons than the reference sequence.

E176. The method of any one of embodiments E171-E175, wherein the product comprises an RNA, e.g., mRNA or an RNA that can be translated.
E177. The method of embodiment E176, wherein the product comprises polypeptide.
E178. The method of any one of embodiments E171-E177, comprising treating a disease or disorder in a subject, by providing an effective amount of a preparation of the product.
E179. The method of any one of embodiments E171-E178, wherein the product comprises RNA.
E180. The method of embodiment E179, wherein the RNA comprises a modification which prevents, e.g., inhibits, the degradation of the RNA, or a modification that alters RNA localization.
E181. The method of embodiment E179 or E180, wherein the modification comprises one or more atoms of a pyrimidine nucleobase replaced or substituted with amino or thiol or a different substitution, e.g., as described in WO 2014/152027 A1.
E182. The method of any one of embodiments E179-E181, wherein the RNA comprises a 5′UTR, a 3′ UTR, a poly A tail, and/or a binding site for a micro RNA.
E183. The method of any one of embodiments E179-E182, wherein the RNA comprises a targeting moiety which targets the RNA to a target tissue or cell.
E184. The method of any one of embodiments E179-E183, wherein the RNA is disposed in a delivery entity, e.g., a nanoparticle, liposome, or a delivery entity described herein.
E185. The method of any one of embodiments E179-E184, wherein the disease or disorder is associated with a con-rare codon, e.g., with a gene having a con-rare codon.
E186. The method of any one of embodiments E179-E185, wherein the con-modified nucleic acid sequence corresponds to the gene having the con-rare codon.
E187. The method of any one of embodiments E179-E186, wherein the product is purified, e.g., by a method described herein.
E188. The method of any one of embodiments E179-E187, wherein the product is a pharmaceutical preparation.
E189. The method of any one of embodiments E179-E188, wherein the product has a production parameter that is different from a production parameter of a product produced from a reference nucleic acid sequence.
E190. The method of embodiment E189, wherein the production parameter is an expression parameter (e.g., as described herein) or a signaling parameter (e.g., as described herein).
E191. The method of any one of embodiments E179-E190, wherein the product is an RNA, e.g., an mRNA, which results in increased expression the nucleic acid sequence having the con-rare codon, e.g., as compared to a reference sequence.
E192. The method of embodiment E189 or E191, wherein the reference sequence is a parental sequence, a starting sequence, a wildtype sequence or a conventionally optimized sequence.
E193. The method of embodiment E192, wherein the reference sequence is an RNA sequence, e.g., a wildtype mRNA sequence or an mRNA sequence transcribed from a conventionally optimized DNA sequence.
E194. A method for identifying a codon-value for a nucleic acid sequence for expression in a target cell or tissue, comprising:

optionally acquiring a nucleic acid sequence from the target cell or tissue;

a) acquiring a codon-value which is a function of the contextual rareness (“con-rarity”) of a sequence-codon in a sequence, wherein the con-rarity of a codon is a function of one or more of the following factors, e.g., by evaluating one or more of the following factors:

(1) the sequence of the codon;

(2) the availability of a corresponding tRNA, e.g., charged tRNA, for that con-rare codon in a target cell or tissue, e.g., one or more isoacceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the t RNA corresponding to the con-rare codon,

b) memorializing, e.g., recording, the codon-value on a medium, e.g., in a machine readable medium,

thereby identifying the codon-value for the nucleic acid sequence.

E195. The method of embodiment E194, wherein b) comprises memorializing the codon-value of a codon in a sequence of codons for the nucleic acid sequence recorded on the medium.
E196. The method of embodiment E194 or E195, wherein the codon-value is the identity of a replacement-codon selected to replace the sequence-codon.
E197. The method of any one of embodiments E194-E196, wherein:

the replacement-codon is a con-abundant codon; and/or

the sequence-codon is a con-rare codon.

E198. The method of any one of embodiments E194-E197, wherein:

the replacement-codon is a con-rare codon; and or

the sequence-codon is a con-abundant codon.

E199. The method of any one of embodiments E194-E198, wherein the replacement-codon is the same as the sequence-codon, e.g., in the case where the sequence-codon is con-abundant and the same con-abundant codon is selected to be the replacement-codon.
E200. The method of any one of embodiments E194-E198, wherein the replacement-codon is the same as the sequence-codon, e.g., in the case where the sequence-codon is con-rare and a con-rare codon is selected to be the replacement-codon.
E201. The method of any one of embodiments E194-E200, comprising repeating step a) and optionally step b) for a second sequence codon.
E202. The method of any one of embodiments E194-E201, comprising repeating step a) and optionally step b) for all of the sequence-codons in a sequence.
E203. The method of any one of embodiments E194-E202, comprising repeating step a) and optionally step b) for an N^{th} sequence codon, wherein N is equal to 3, 4, 5, 6, 7, 8, 9, 10, 11, 12, 13, 14, 15, 16, 17, 18, 19, 20, 25, 30, 35, 40, 45, 50, 100, 150, 200, 300, 400, 500, 600, 700, 800, 900 or 1000.
E204. The method of any one of embodiments E194-E203, wherein responsive to said codon-value, altering the memorialized (e.g., recorded) nucleic acid sequence by replacing at least 0, 1, 2, 3, 4, 5, 10, 15, 20, 30, 50, 100 or all of the con-rare codons in the memorialized sequence with a con-abundant codon in the target cell or tissue.
E205. The method of any one of embodiments E194-E204, wherein responsive to said codon-value, altering the memorialized (e.g., recorded) nucleic acid sequence by replacing at least 0, 1, 2, 3, 4, 5, 10, 15, 20, 30, 50, 100 or all of the con-abundant codons in the memorialized sequence with a con-rare codon in the target cell or tissue.
E206. The method of embodiment E205, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of the con-rare codons with a con-abundant codon in the target cell or tissue.
E207. The method of embodiment E204 or E206, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of the con-rare codons with a con-abundant codon in the target cell or tissue.
E208. The method of embodiment E207, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of con-abundant codons with a con-rare codon in the target cell or tissue.
E209. The method of embodiment E206 or E208, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of con-abundant codons with a con-rare codon in the target cell or tissue.
E210. The method of any one of embodiments E194-E209, further comprising synthesizing a nucleic acid sequence comprising one or more of the replacement-codons to provide a contextually-modified (“con-modified”) nucleic acid sequence.
E211. The method of any one of embodiments E194-E210, further comprising forming a bond between a first nucleotide and a second nucleotide to provide a contextually-modified (“con-modified”) nucleic acid sequence from the memorialized codon-value provided in embodiment E186(b).
E212. The method of any one of embodiments E194-E211, which is computer implemented.
E213. A method for making a contextually modified (“con-modified”) nucleic acid sequence for expression in a target cell or tissue, comprising:

optionally acquiring a nucleic acid sequence from the target cell or tissue;

a) acquiring a codon-value which is a function of the contextual rareness (“con-rarity”) of a sequence-codon in a sequence, wherein the con-rarity of a codon is a function of one or more of the following factors, e.g., by evaluating one or more of the following factors:

(1) the sequence of the codon;

(2) the availability of a corresponding tRNA, e.g., charged tRNA, for that con-rare codon in a target cell or tissue, e.g., one or more isoacceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon,

b) memorializing, e.g., recording, the codon-value on a medium, e.g., in a machine readable medium;

c) responsive to said codon-value, altering the memorialized (e.g., recorded) nucleic acid sequence by:

(i) replacing at least 0, 1, 2, 3, 4, 5, 10, 15, 20, 30, 50, 100 or all of the con-rare codons in the memorialized sequence with a con-abundant codon in the target cell or tissue; and/or

(ii) replacing at least 0, 1, 2, 3, 4, 5, 10, 15, 20, 30, 50, 100 or all of the con-abundant codons in the memorialized sequence with a con-rare codon in the target cell or tissue; and/or

d) synthesizing a nucleic acid sequence comprising one or more of the replacement-codons to provide a contextually-modified (“con-modified”) nucleic acid sequence,

thereby making a con-modified nucleic acid sequence for expression in a target cell or tissue.

E214. The method of embodiment E213, comprising replacing at least 0, 1, 2, 3, 4, 5, 10, 15, 20, 30, 50, 100 or all of the con-rare codons in the memorialized sequence with a con-abundant codon in the target cell or tissue.
E215. The method of embodiment E213, comprising replacing at least 0, 1, 2, 3, 4, 5, 10, 15, 20, 30, 50, 100 or all of the con-abundant codons in the memorialized sequence with a con-rare codon in the target cell or tissue.
E216. The method of any one of embodiments E210-E215, wherein the synthesized sequence has at least 50%, 60%, 70%, 80%, 90% or 100% identity to the starting sequence, e.g., reference sequence.
E217. The method of any one of embodiments E210-E216, wherein the synthesized sequence has at least 50%, 60%, 70%, 80%, 90% or 100% of the number of codons in the starting sequence, e.g., reference sequence.
E218. The method of any one of embodiments E210-E217, wherein the synthesized sequence encodes for a protein or polypeptide having at least 50%, 60%, 70%, 80%, 90% or 100% identity to the protein or polypeptide encoded by the starting sequence, e.g., reference sequence.
E219. The method of any one of embodiments E210-E218, wherein the synthesized sequence encodes for a polypeptide or protein having a substantially similar property, e.g., function and/or expression level, as a polypeptide or protein encoded by the starting sequence, e.g., reference sequence.
E220. The method of any one of embodiments E210-E219, wherein the synthesized sequence results in a messenger RNA having a substantially similar property, e.g., function and/or expression level, as a messenger RNA made by the starting sequence, e.g., reference sequence.
E221. A method for modifying a nucleic acid sequence, comprising:

acquiring a nucleic acid sequence, e.g., a nucleic acid sequence which is expressed in a target cell or tissue, or a nucleic acid sequence which is not expressed in a target cell or a tissue;

determining the presence of a contextually-rare codon (“con-rare codon”) in said sequence;

replacing at least 0, 1, 2, 3, 4, 5, 10, 15, 20, 30, 50, 100 or all of the con-rare codons with a different codon, e.g., a con-abundant codon; and/or replacing at least 0, 1, 2, 3, 4, 5, 10, 15, 20, 30, 50, 100 or all of the non-con-rare codons (con-abundant codon) with a con-rare codon.

E222. The method of embodiment E221, which is computer implemented.
E223. The method of embodiment E221 or E222, wherein determining the presence of a contextually-rare codon (“con-rare codon”) in said sequence comprises evaluating one or more of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon.

E224. The method of any one of embodiments E221-E223, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of the con-rare codons with a con-abundant codon in the target cell or tissue.
E225. The method of any one of embodiments E221-E224, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of the con-rare codons with a con-abundant codon in the target cell or tissue.
E226. The method of any one of embodiments E221-E225, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of the con-abundant codons with a con-rare codon in the target cell or tissue.
E227. The method of any one of embodiments E221-E226, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of the con-abundant codons with a con-rare codon in the target cell or tissue.
E228. A system for identifying a codon-value for a nucleic acid sequence for expression in a target cell or tissue, comprising:

a computer, e.g., a general purpose computer, configured for:

optionally, acquiring a nucleic acid sequence from the target cell or tissue;

a) acquiring, e.g., calculating, a codon-value which is a function of the contextual-rareness (“con-rarity”) of a sequence-codon in a sequence, wherein the con-rarity of a codon is a function of one or more of the following factors, e.g., by evaluating one or more of the following factors:

(1) the sequence of the codon;

(2) the availability of a corresponding tRNA, e.g., charged tRNA, for that con-rare codon in a target cell or tissue, e.g., one or more isoacceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the t RNA corresponding to the con-rare codon,

b) memorializing, e.g., recording, the codon-value on a medium, e.g., in a machine readable medium,

thereby identifying the codon-value for the nucleic acid sequence.

E229. The system of embodiment E228, wherein b) comprises memorializing the codon-value of a codon in a sequence of codons for the nucleic acid sequence recorded on the medium.
E230. The system of embodiment E228 or E229, wherein the codon-value is the identity of a replacement-codon selected to replace the sequence-codon.
E231. The system of any one of embodiments E228-E230, wherein:

the replacement-codon is a con-abundant codon; and/or

the sequence-codon is a con-rare codon.

E232. The system of any one of embodiments E228-E231, wherein:

the replacement-codon is a con-rare codon; and or

the sequence-codon is a con-abundant codon.

E233. The system of any one of embodiments E228-E232, wherein the replacement-codon is the same as the sequence-codon, e.g., in the case where the sequence-codon is con-abundant and the same con-abundant codon is selected to be the replacement-codon.
E234. The system of any one of embodiments E228-E233, wherein the replacement-codon is the same as the sequence-codon, e.g., in the case where the sequence-codon is con-rare and a con-rare codon is selected to be the replacement-codon.
E235. The system of any one of embodiments E228-E234, comprising repeating step a) and optionally step b) for a second sequence codon.
E236. The system of any one of embodiments E228-E235, comprising repeating step a) and optionally step b) for all of the sequence-codons in a sequence.
E237. The system of any one of embodiments E228-E236, comprising repeating step a) and optionally step b) for an N^{th} sequence codon, wherein N is equal to 3, 4, 5, 6, 7, 8, 9, 10, 11, 12, 13, 14, 15, 16, 17, 18, 19, 20, 25, 30, 35, 40, 45, 50, 100, 150, 200, 300, 400, 500, 600, 700, 800, 900 or 1000.
E238. The system of any one of embodiments E228-E237, wherein responsive to said codon-value, altering the memorialized (e.g., recorded) nucleic acid sequence by replacing at least 0, 1, 2, 3, 4, 5, 10, 15, 20, 30, 50, 100 or all of the con-rare codons in the memorialized sequence with a con-abundant codon in the target cell or tissue.
E239. The system of any one of embodiments E228-E238, wherein responsive to said codon-value, altering the memorialized (e.g., recorded) nucleic acid sequence by replacing at least 0, 1, 2, 3, 4, 5, 10, 15, 20, 30, 50, 100 or all of the con-abundant codons in the memorialized sequence with a con-rare codon in the target cell or tissue.
E240. The system of embodiment E239, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of the con-rare codons with a con-abundant codon in the target cell or tissue.
E241. The system of embodiment E238 or E240, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of the con-rare codons with a con-abundant codon in the target cell or tissue.
E242. The system of embodiment E241, which comprises replacing at least 1%, 2%, 3%, 5%, 10%, 15%, 20%, 25%, 30%, 35%, 40%, 45%, 50%, 55%, 60%, 65%, 70%, 75%, 80%, 85%, 95%, 99% or all of con-abundant codons with a con-rare codon in the target cell or tissue.
E243. The system of embodiment E238 or E242, which comprises replacing about 1% to 99%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 2% to 99%, 3% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, or 95% to 99%, of con-abundant codons with a con-rare codon in the target cell or tissue.
E244. The system of any one of embodiments E228-E243, further comprising synthesizing a nucleic acid sequence comprising one or more of the replacement-codons to provide a contextually-modified (“con-modified”) nucleic acid sequence.
E245. The system of any one of embodiments E228-E244, further comprising forming a bond between a first nucleotide and a second nucleotide to provide a con modified nucleic acid sequence from the memorialized codon-value provided in embodiment E220(b).
E246. The system of any one of embodiments E228-E245, which is computer implemented.

Other features, objects, and advantages of the disclosure will be apparent from the description and from the claims.

Unless otherwise defined, all technical and scientific terms used herein have the same meaning as commonly understood by one of ordinary skill in the art to which this disclosure belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the disclosure may be better understood when read in conjunction with the appended drawings. It should be understood, however, that the disclosure is not limited to the precise arrangement and instrumentalities of the embodiments shown in the drawings.

FIGS. 1A-1B are images depicting the tRNA levels in HEK293T cells as quantified by Oxford Nanopore sequencing, as described in Example 1. FIG. 1A depicts tRNA profiling by Nanopore sequencing, wherein each line in the graph demonstrates a different sample preparation method. FIG. 1B depicts the levels of tRNA in normal cells compared to cells overexpressing the iMet tRNA.

FIG. 2 depicts the contextual rarity of tRNAs in HEK293T cells. The x axis shows the tRNA frequency in HEK293T cells as determined by tRNA quantification and the y axis shows the HEK293T proteome codon count as determined by the sum of all protein codon counts multiplied by the protein's respective abundance.

FIG. 3 depicts the increased fold-change in expression of 8 genes (APLN, APOA2, APOC2, APOC3, BET1, CCL3, CENPX, and GNGT2) as measured in mammalian lysate, when the sequence of these genes is contextually modified using three different methods as comparted to the wild type sequence.

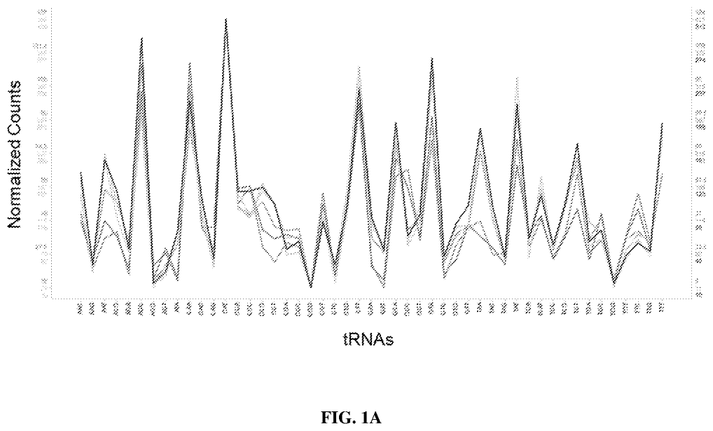

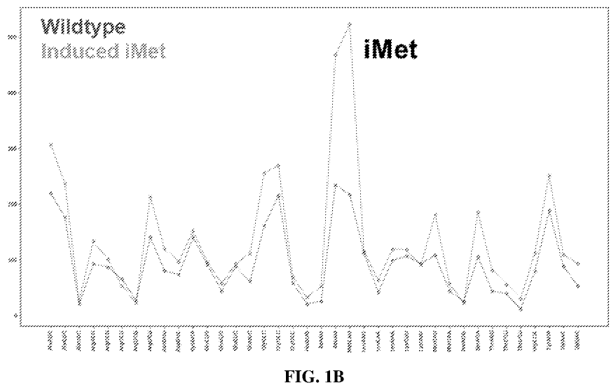

### DETAILED DESCRIPTION OF CERTAIN EMBODIMENTS

The present disclosure features, inter alia, the observation that codon identity and function is in fact highly contextual and varied within an organism. Accordingly, this disclosure provides, inter alia, methods of identifying a contextually rare codon (“con-rare codon”), methods of modifying a nucleic acid sequence comprising one or a plurality of con-rare codons, and uses of said methods.

### Definitions

As used herein, the articles “a” and “an” refer to one or to more than one (e.g., to at least one) of the grammatical object of the article.

A “contextually rare codon” or “con-rare codon”, as those terms are used herein, refer to a codon which, in a target cell or tissue, is limiting for a production parameter, e.g., an expression parameter, for a nucleic acid sequence having a con-rare codon (“con-rare codon nucleic acid sequence”), e.g., because the availability of a tRNA corresponding to the con-rare codon is limiting for a production parameter. Contextual rareness or con-rarity can be identified or evaluated by determining if the addition of a tRNA corresponding to a con-rare codon modulates, typically increases, a production parameter for a nucleic acid sequence, e.g., gene. Contextual rareness or con-rarity can be identified or evaluated by whether a codon satisfies a reference value for protein codon count-tRNA frequency (PCC-tF, as described herein). By way of example, the method of Example 3, can be used, or adapted to be used, to evaluate con-rarity. Con-rarity as a property of a codon, is a function of, and can be identified or evaluated on the basis of, one, two, three, four, five, six, or all of the following factors:

(1) the sequence of the con-rare codon, or candidate con-rare codon;

(2) the availability of a corresponding tRNA for the con-rare, or candidate con-rare, -codon in a target cell or tissue Availability as a parameter can comprise or be a function of, one or both of the observed or predicted abundance or availability of a tRNA that corresponds to the con-rare codon. In an embodiment, abundance can be evaluated by quantifying tRNAs present in a target cell or tissue. See, e.g., Example 1a-1b;

(3) the contextual demand (the demand in a target cell or tissue) for a tRNA, e.g., a con-rare tRNA, or a candidate con-rare tRNA. This can be identified or evaluated by use of a parameter, a contextual demand-parameter, which comprises or is a function of, the demand or usage of a con-rare tRNA by one, some, or all of the nucleic acid sequences having con-rare codons in a target tissue or cell, e.g., the other nucleic acid sequences in a target cell or tissue which have a con-rare codon. A demand parameter can comprise of, or be a function of one or more, or all of:
(a) the expression profile (or proteomic properties) in the target cell or tissue (e.g., the abundance of expression) of other nucleic acid sequences in the target cell or tissue which have a con-rare codon (e.g., for one or more, a subset of, or all of the expressed con-rare codon nucleic acid sequences in the target cell or tissue). In an embodiment, the expression profile (or proteomic properties) can be evaluated by evaluating proteins expressed in a target cell or tissue. See, e.g., Example 2;
(b) a measure which comprises or is a function of the frequency or proportion of appearance of the con-rare codon in an expressed nucleic acid sequence (e.g., for one or more, a subset of, or all of the expressed con-rare codon nucleic acid sequences in the target cell or tissue); or
(c) a parameter that is a function of (3)(a) and (3)(b);

(4) a parameter (or use-parameter) related to the con-rare codon usage in a con-rare codon nucleic acid sequence, and can include one or more of:
(a) the expression profile (or proteomic properties) in the target cell or tissue (e.g., the abundance of expression) of other nucleic acid sequences in the target cell or tissue which have a con-rare codon, or a candidate nucleic acid sequence having a con-rare codon, (e.g., for one or more, a subset of, or all of the expressed con-rare codon nucleic acid sequences(s) in the target cell or tissue). In an embodiment, the expression profile (or proteomic properties) can be evaluated by evaluating proteins expressed in a target cell or tissue. See, e.g., Example 2;
(b) a measure which comprises or is a function of the frequency or proportion of appearance of the con-rare codon in a nucleic acid sequence having a con-rare codon (e.g., for one or more, a subset of, or all of the expressed con-rare codon nucleic acid sequence(s) in the target cell or tissue); or
(c) a parameter that is a function of (4)(a) and (4)(b);

(5) the proportion of the tRNAs corresponding to the con-rare codon which are charged;

(6) the iso-decoder isotype of the tRNA corresponding to the con-rare codon; and

(7) one or more post-transcriptional modifications of the con-rare tRNA, or candidate con-rare tRNA; and

In an embodiment, a con-optimized nucleic acid sequence has one less or one more con-rare codon than a reference sequence, e.g., a parental sequence, a naturally occurring sequence, a wildtype sequence, or a conventionally optimized sequence.

In an embodiment, con-rarity can be identified or evaluated by: (i) direct determination of whether a con-rare codon or candidate con-rare codon is limiting for a production parameter, e.g., in an assay analogous to that of Example 3; (ii) whether a con-rare or candidate con-rare codon meets a predetermined value, e.g., a standard or reference value (e.g., as described herein), of one or more, or all of factors (1)-(7); or (i) and (ii).

In an embodiment, con-rarity can be identified or evaluated by a production parameter, e.g., an expression parameter or a signaling parameter, e.g., as described herein.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell.

Thus, the identification of a codon as a con-rare codon can involve a multi-parameter function of (1)-(7). In an embodiment, the con-rare codon meets a reference value for at least one of (1)-(7). In an embodiment, the con-rare codon meets a reference value for at least one of (1)-(7). In an embodiment, the con-rare codon meets a reference value for at least two of (1)-(7). In an embodiment, the con-rare codon meets a reference value for at least three of (1)-(7). In an embodiment, the con-rare codon meets a reference value for at least four of (1)-(7). In an embodiment, the con-rare codon meets a reference value for at least five of (1)-(7). In an embodiment, the con-rare codon meets a reference value for at least six of (1)-(7). In an embodiment, the con-rare codon meets a reference value for at all of (1)-(7). In an embodiment the reference value is a pre-determined or pre-selected value, e.g., as described herein.

In an embodiment, the identity of a con-rare codon is the DNA sequence which encodes for the codon in the nucleic acid sequence, e.g., gene.

In an embodiment, a con-rare codon is other than an iMet codon.

The methods disclosed herein, e.g., in the examples, provided herein, can be used to identify and test candidate con-rare codons.

In an embodiment, a con-rare codon is a function of the prevalence of the codon in the open reading frame (ORF) of protein coding genes in an organism, e.g., the proteome.

The availability, e.g., abundance, of tRNAs that correspond to a con-rare codon can be measured using an assay known in the art or as described herein, e.g., Nanopore sequencing, e.g., as described in Example 1. In an embodiment, a con-rare codon nucleic acid sequence has a low abundance of a tRNA corresponding to the con-rare codon, e.g., as compared to the abundance of a tRNA corresponding to a different/second codon.

The expression profile or proteomic property of a target cell or tissue refers to the protein expression, e.g., level of protein expression, from all of the protein coding genes in a target cell or tissue. The expression profile or proteomic property of a target cell or tissue can be measured using an assay known in the art or as described herein, e.g., a mass spectrometry based method, e.g., a SILAC based method as described in Example 2. In an embodiment, a protein coding gene in a target cell or tissue is a function of tissue or cell type specific regulation, e.g., a promoter element, an enhancer element, epigenetic regulation, and/or transcription factor control.

A “contextually-modified nucleic acid sequence” (sometimes referred to herein as a “con-modified nucleic acid sequence”) refers to a nucleic acid sequence in which the con-rarity of a codon of the con-modified nucleic acid sequence has been altered. E.g., a con-rare codon is replaced with a con-abundant codon and/or a con-abundant codon is replaced with a con-rare codon. In an embodiment, the con-modified nucleic acid sequence has one more or one less, e.g., two more or two lesser, con-rare codons, than a reference nucleic acid sequence. In an embodiment, the con-modified nucleic acid sequence has a codon with con-rarity that differs from the con-rarity of the corresponding codon in a reference nucleic acid sequence.

The reference nucleic acid sequence can be, e.g., any selected sequence, a parental sequence, a starting sequence, a wildtype or naturally occurring sequence that encodes the same amino acid at the corresponding codon, a wildtype or naturally occurring sequence that encodes the same polypeptide, or a conventionally codon-optimized sequence. In an embodiment, the reference nucleic acid sequence encodes the same polypeptide sequence as the con-modified nucleic acid sequence. In an embodiment, the reference nucleic acid sequence encodes a polypeptide sequence that differs from the con-modified nucleic acid sequence at a position other than the con-rare modified sequence. In an embodiment, a con-modified nucleic acid sequence results in a different production parameter, e.g., an expression parameter or signaling parameter, compared to that seen with expression of a reference nucleic acid sequence.

In an embodiment, a con-modified nucleic acid sequence refers to a nucleic acid sequence which has one more or one less, e.g., two more or two lesser, con-rare codons, than a reference sequence, wherein the con-modified nucleic acid sequence encodes a polypeptide that comprises the reference sequence.

A “contextually-rare tRNA” or “con-rare tRNA,” is a tRNA that corresponds to a con-rare codon.

A “con-rare codon nucleic acid sequence,” or a “nucleic acid sequence having a con-rare codon” as those terms are used herein, refer to a nucleic acid sequence, e.g., gene, comprising a con-rare codon. In an embodiment, in such con-rare codon nucleic acid sequences, modulation of an expression parameter can be mediated by altering the availability, e.g., abundance of a con-rare tRNA. In an embodiment, the con-rare codon is in a translated region of the con-rare codon nucleic acid sequence, e.g., in an open reading frame (ORF) or coding sequence (CDS).

A “contextually-abundant codon” or “con-abundant codon” as those terms are used herein, refer to a codon other than a con-rare codon.

A “codon-value” as that term is used herein, is a function of the con-rarity of a sequence-codon in a sequence. Con-rarity of a codon is a function of one or more factors as described in the definition of “con-rare codon” above. In an embodiment, a codon-value is the identity of a codon, e.g., a replacement codon selected to replace the sequence-codon. In an embodiment, when the replacement codon is a con-abundant codon, the sequence codon is a con-rare codon. In an embodiment, when the replacement codon is a con-rare codon, the sequence-codon is a con-abundant codon.

A “sequence-codon” as that term is used herein, refers to a codon in a nucleic acid sequence for which a codon-value is acquired.

A “production parameter,” refers to an expression parameter and/or a signaling parameter. In an embodiment a production parameter is an expression parameter. An expression parameter includes an expression parameter of a polypeptide or protein encoded by the con-rare codon nucleic acid sequence; or an expression parameter of an RNA, e.g., messenger RNA, encoded by the con-rare codon nucleic acid sequence. In an embodiment, an expression parameter can include:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

In an embodiment, a production parameter is a signaling parameter. A signaling parameter can include:

(1) modulation of a signaling pathway, e.g., a cellular signaling pathway which is downstream or upstream of the protein encoded by the con-rare codon nucleic acid sequence;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

A “decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced.

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a con-modified nucleic acid sequence or a product of a con-modified nucleic acid sequence described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

An “increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

The terms modified, replace, derived and similar terms, when used or applied in reference to a product, refer only to the end product or structure of the end product, and are not restricted by any method of making or manufacturing the product, unless expressly provided as such in this disclosure.

Headings, titles, subtitles, numbering or other alpha/numeric hierarchies are included merely for ease of reading and absent explicit language to the contrary do not indicate order of performance, order of importance, magnitude or other value.

### Contextually-Rare Codons and Methods of Identifying a Con-Rare Codon

Disclosed herein is the observation that codon identity and function is in fact highly contextual and varied within an organism. Accordingly, this disclosure provides, inter alia, methods of identifying a contextually rare codon (“con-rare codon”), methods of modifying a nucleic acid sequence comprising one or a plurality of con-rare codons and uses of said methods.

A con-rare codon is a codon that is limiting for a production parameter, e.g., an expression parameter or a signaling parameter, for a nucleic acid sequence, e.g., gene. Contextual rareness or con-rarity can be identified or evaluated by determining if the addition of a tRNA corresponding to a con-rare codon modulates, typically increases, a production parameter for a nucleic acid sequence, e.g., gene. In an embodiment, con-rarity as a property of a codon, is a function of, one, two, three, four, all of the following factors:

(1) the sequence of the codon;

(2) the availability of a corresponding tRNA, e.g., charged tRNA, for that con-rare codon in a target cell or tissue, e.g., one or more iso-acceptor tRNA molecules;

(3) the expression profile (or proteomic properties) of the target cell or tissue (e.g., the abundance of expression of other proteins which include the con-rare codon);

(4) the proportion of the tRNAs corresponding to the con-rare codon which are charged; and

(5) the iso-decoder isotype of the tRNA corresponding to the con-rare codon.

In an embodiment, con-rarity is a function of normalized proteome codon count and tRNA abundance in a target tissue or cell. In an embodiment, con-rarity is a measure of codon frequency that is contextually dependent on tRNA abundance levels in a target tissue or cell. In an embodiment, con-rarity can be identified or evaluated by a production parameter, e.g., an expression parameter or a signaling parameter, e.g., as described herein.

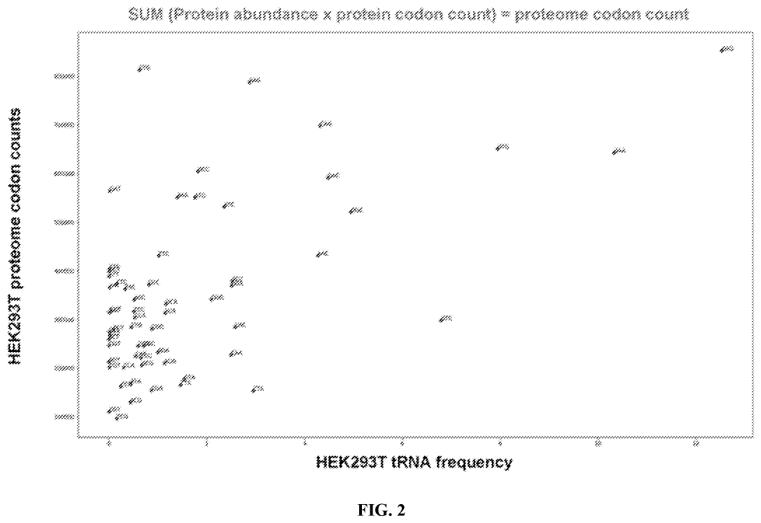

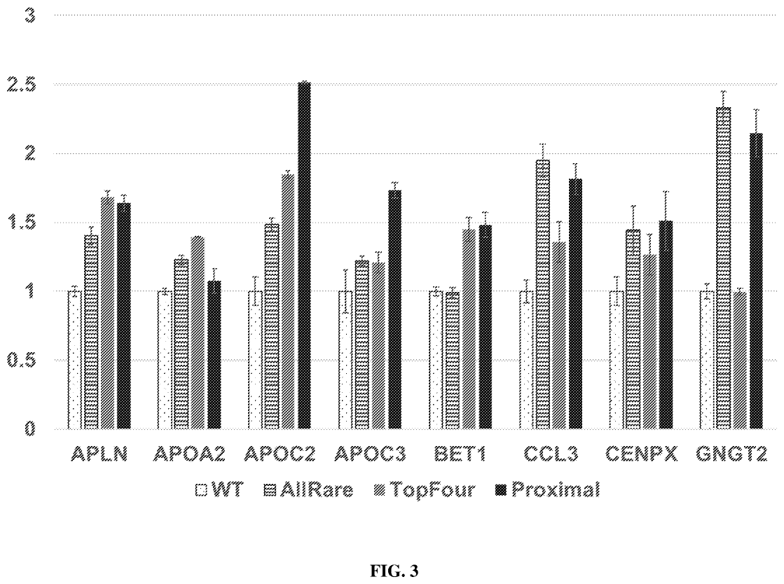

An exemplary method of evaluating con-rarity and identifying a con-rare codon is provided in Example 3, or for example, FIG. 2.

### Exemplary Reference Values for Evaluating Con-Rarity

In an embodiment, contextual rareness or con-rarity can be identified or evaluated by whether a codon satisfies a reference value for protein codon count-tRNA frequency (PCC-tF, as described herein).

In an embodiment, con-rarity is a function of normalized proteome codon count and the tRNA profile, e.g., as described herein. In an embodiment, con-rarity is determined by dividing the normalized proteome codon count by the tRNA profile determined by Nanopore or other tRNA sequencing experiment. This provides a measure of codon usage that is contextually dependent on the tRNA profile, e.g., tRNA abundance levels.

In an embodiment, a codon is determined to be contextually rare (con-rare) if the con-rarity meets a reference value, e.g., a pre-determined or pre-selected reference value, e.g., a threshold, e.g., an internal threshold, e.g., as described herein. In an embodiment, the reference value is a value under which e.g., 1.5× sigma of the normally fit distribution to that codon frequency.

In an embodiment, a codon is con-rare if the value of a normalized proteome codon count divided by the tRNA profile value for a particular tRNA meets a reference value, e.g., a pre-determined or pre-selected reference value, e.g., a threshold, e.g., an internal threshold.

In an embodiment, a codon is con-rare if the value of a normalized proteome codon count divided by the tRNA profile value for a particular tRNA is in the top 5%, 10%, 20%, 30%, or 40% of values for normalized proteome codon count divided by the tRNA profile value for all codons measured, e.g., wherein all 64 codons are measured. In an embodiment, a codon is con-rare if the value of a normalized proteome codon count divided by the tRNA profile value for a particular tRNA is in the top 5% of values for normalized proteome codon count divided by the tRNA profile value for all codons measured. In an embodiment, a codon is con-rare if the value of a normalized proteome codon count divided by the tRNA profile value for a particular tRNA is in the top 10% of values for normalized proteome codon count divided by the tRNA profile value for all codons measured. In an embodiment, a codon is con-rare if the value of a normalized proteome codon count divided by the tRNA profile value for a particular tRNA is in the top 20% of values for normalized proteome codon count divided by the tRNA profile value for all codons measured. In an embodiment, a codon is con-rare if the value of a normalized proteome codon count divided by the tRNA profile value for a particular tRNA is in the top 30% of values for normalized proteome codon count divided by the tRNA profile value for all codons measured. In an embodiment, a codon is con-rare if the value of a normalized proteome codon count divided by the tRNA profile value for a particular tRNA is in the top 40% of values for normalized proteome codon count divided by the tRNA profile value for all codons measured.

In an embodiment, a codon is con-rare if for the value of a normalized proteome codon count divided by the tRNA profile value for a particular tRNA, the value for the normalized proteome codon count is below the value for all codons measured and the value for tRNA profile, is above the value for all codons measured, e.g., wherein all 64 codons are measured.

In an embodiment, a codon is a con-rare codon if it is in the upper left quadrant of a plot of normalized proteome codon count (y-axis) vs tRNA profile (x-axis), with equal number of codons in each quadrant, e.g., wherein all 64 codons are measured.

In an embodiment, a codon is a con-rare codon if it is in a quadrant other than the lower right quadrant of a plot of normalized proteome codon count (y-axis) vs tRNA profile (x-axis), with equal number of codons in each quadrant, e.g., wherein all 64 codons are measured.

### Proteome Codon Count-tRNA Frequency (PCC-tF)

In another aspect, proteome codon count (for a selected codon) can be used in conjunction with tRNA frequency (for tRNAs having the selected codon) to provide a measure of con-rarity for the selected codon. This parameter is referred to herein as proteome codon count-tRNA frequency, or PCC-tF. Proteome codon count can serve as a measure of “demand” for a tRNA having a selected codon. tRNA frequency can serve as a measure of “supply” for a tRNA having a selected codon.

Proteome codon count, as used herein, refers to the sum (for all of the proteins of a set of reference proteins in a target cell (or tissue)) of the number of times the codon is used in a protein of the reference set multiplied by the value of that protein's abundance. Proteome codon count can be expressed as Σ(protein abundance x protein codon count)_{R1-Rn}, wherein R is the set of proteins. Typically the reference set is all of the proteins expressed in a target cell (or tissue) or a portion of the proteins expressed in a target cell, e.g., all proteins for which the abundance of the protein is greater than 1%, 5%, 10%, 15%, 20%, 25%, 30%, 35%, 40%, 45%, 50%, 55%, 60%. 65%, 70%, 75%, 80%, 85%, 90%, 95% or more by number or molecular weight of all the proteins expressed in the target cell (or tissue) or all of the proteins detectable by a method to determine proteomic quantification, e.g., mass spectrometry.

tRNA frequency for a selected target cell (or tissue) can be determined, by way of example, by sequencing methods.

Con-rarity, (or an element of con-rarity, where other elements contribute to the overall determination of con-rarity), for a codon can be defined or evaluated by a function of a codon's proteome codon count and its cognate tRNA frequency in a target cell (or tissue), e.g, by the a function of the ratio of one to the other (PCC-tF). In an embodiment, the function is the ratio of tRNA frequency to proteome codon count. If increasing tRNA frequency is plotted on the x axis and increasing proteome codon count is plotted on the Y axis (see, e.g., FIG. 2) then in an embodiment, the tendency toward the upper left quadrant is associated with relatively greater con-rarity and the tendency toward the bottom right quadrant is associated with relatively lessor con-rarity.

Con-rarity, (or an element of con-rarity), for a codon can be defined or evaluated by the codon satisfying a reference value for proteome codon count and satisfying a reference value for tRNA frequency in a target cell (or tissue), or for satisfying a reference value for PCC-tF.

The range of values for proteome codon count for a set of reference proteins can be divided into subranges, e.g., into quartiles, quintiles, deciles, or percentiles. Likewise, the range of values for tRNA frequency (for a selected codon) can divided into subranges, e.g., into quartiles, quintiles, deciles, or percentiles. In an embodiment, con-rarity (or an element of con-rarity) can be defined or evaluated as a codon which meets a selected reference for proteome codon count and meets a selected reference for tRNA frequency.

In an embodiment, a codon is con-rare (or satisfies an element of con-rarity) if the codon falls within a selected subrange or set of subranges for proteome codon count and has a codon frequency of less than a reference value or which falls into a selected subrange or set of subranges for frequency, or has a value for PCC-tF corresponding to satisfying such selected subranges or sets of subranges.

In an embodiment, a codon is con-rare (or satisfies an element of con-rarity) if it is in fifth decile or above for proteome codon count and in the fifth decile, or lower, for tRNA frequency, or has a value for PCC-tF corresponding to satisfying such selected subranges or sets of subranges.

In an embodiment, a codon is con-rare (or satisfies an element of con-rarity) if it is in fourth decile or above for proteome codon count and in the fourth decile, or lower, for tRNA frequency, or has a value for PCC-tF corresponding to satisfying such selected subranges or sets of subranges.

In an embodiment, a codon is con-rare (or satisfies an element of con-rarity) if it is in third decile or above for proteome codon count and in the third decile, or lower, for tRNA frequency, or has a value for PCC-tF corresponding to satisfying such selected subranges or sets of subranges.

In an embodiment, a codon is con-rare (or satisfies an element of con-rarity) if it is in second decile or above for proteome codon count and in the second decile, or lower, for tRNA frequency, or has a value for PCC-tF corresponding to satisfying such selected subranges or sets of subranges.

In an embodiment, a codon is con-rare (or satisfies an element of con-rarity) if it is in first decile or above for proteome codon count and in the first decile, or lower, for tRNA frequency, or has a value for PCC-tF corresponding to satisfying such selected subranges or sets of subranges.

### Con-Modified Nucleic Acid Sequences and Methods of Making the Same

A nucleic acid sequence that is identified to have one or more con-rare codons can be modified by, e.g., replacing, said con-rare codon with a codon other than a con-rare codon, e.g., a con-abundant codon. In an embodiment, a con-modified nucleic acid sequence has an altered con-rarity for a codon. In an embodiment, a con-modified nucleic acid sequence is a sequence in which a con-rare codon is replaced with a con-abundant codon and/or a con-abundant codon is replaced with a con-rare codon. In an embodiment, a con-modified nucleic acid sequence has one more or one less, e.g., two more or two lesser, con-rare codons, than a reference nucleic acid sequence. In an embodiment, the con-modified nucleic acid sequence has a codon with con-rarity that differs from the con-rarity of the corresponding codon in a reference nucleic acid sequence.

A con-modified nucleic acid sequence can be made according to any of the methods disclosed herein. The disclosure also contemplates a computer implemented method for codon-modifying a nucleic acid sequence, e.g., as provided in Example 7.

An exemplary computational algorithm that can be used to codon modify a nucleic acid sequence is provided below:

```
import urllib2
import sys
from collections import defaultdict
from collections import Counter
import os
from Bio import SeqIO
from Bio.Seq import Seq
#Read in codon frequency list
#Codon frequency determined by taking the sum of the number of times a codon appears per
gene multiplied by the number of times that gene appears in a cell (determined by a proteomics
experiment) to give a normalized codon frequency per gene. The sum of the normalized codon
frequency per gene are summed across all genes in that cell line to a give a normalized codon
frequency per cell line. The number is then divided by the normalized tRNA frequency. The
normalized tRNA frequency is determined by taking the aligned read counts per tRNA. The
counts per tRNA are normalized to 1.
codons_by_aa = defaultdict(list)
for 1 in open(“codon_frequency.txt”,“r”):
  codon = l.split(“\t”)[0].strip( )
  amino_acid = Seq(codon).translate( )
  frequency = float(l.split(“\t”)[1])
  codons_by_aa[amino_acid].append((frequency, codon))
#Sort through codon frequency file and create a table of codons to switch out, if there exists
another codon that encodes that amino acid that has a frequency 1.5× times the codon of interest
optimal_switch_dictionary = { }
for amino_acid in codons_by_aa:
  sorted_codon_per_aa = sorted(codons_by_aa[amino_acid])
  if len(sorted_codon_per_aa) > 1:
    max = sorted_codon_per_aa[−1][0]
    max_codon = sorted_codon_per_aa[−1][1]
    for codon in sorted_codon_per_aa:
      if codon[0]*1.5 < max:
        optimal_switch_dictionary[codon[1]] = max_codon
def break_to_codon(string, length):
  codon_list = [ ]
  #count each codon type
  #return Counter(string[0+i:length+i].upper( ) for i in range(0, len(string), length))
  for i in range(0, len(string), length):
    j = string[0+i:length+i].upper( )
    if j in optimal_switch_dictionary:
      codon_list.append(optimal_switch_dictionary[j])
    else:
      codon_list.append(j)
  return codon_list
#Read in FASTA File with sequence to optimize
for seq_record in SeqIO.parse(sys.argv[1], “fasta”):
  seq = seq_record.seq
  #Break sequence into codons and replace those that are nonprime
  codon_list = break_to_codon(str(seq),3)
  print “”.join(codon_list)
```

Other or similar computational algorithms in, e.g., different programming languages, can be used to make con-modified nucleic acid sequences disclosed herein.

### Uses of a Con-Modified Nucleic Acid Sequence

A con-modified nucleic acid sequence disclosed herein can have a modulated, e.g., an altered, production parameter, e.g., an expression parameter or a signaling parameter, which is altered or modulated compared to the corresponding parameter in a parental nucleic acid sequence, e.g., as described herein.

For example, a con-modified nucleic acid sequence can have an increase or decrease in any one or more of the following expression parameters:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

As another example, a con-modified nucleic acid sequence can have an increase or decrease in any one or more of the following signaling parameters:

(1) modulation of a signaling pathway, e.g., a cellular signaling pathway which is downstream or upstream of the protein encoded by the con-rare codon nucleic acid sequence;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

A production parameter (e.g., an expression parameter and/or a signaling parameter) may be modulated, e.g., by at least 5% (e.g., at least 10%, 15%, 20%, 25%, 30%, 40%. 50%. 60%. 70%, 80%, 90%, 100%, 150%, 200% or more) compared to a reference nucleic acid sequence, e.g., parental, wildtype or conventionally optimized nucleic acid sequence.

In an embodiment, a con-modified nucleic acid sequence, e.g., a composition or pharmaceutical composition comprising a con-modified nucleic acid sequence, can be used for a use described herein. For example, a composition or pharmaceutical composition comprising a con-modified nucleic acid sequence can modulate a function in a cell, tissue or subject.

In an embodiment, a con-modified nucleic acid sequence can be used to manufacture a product of said con-modified nucleic acid sequence, e.g., a protein, polypeptide or RNA which can be translated into a protein of polypeptide. In an embodiment, the manufacturing can be performed in vivo, in vitro or ex vivo. For example, in vitro manufacturing of a product of a con-modified nucleic acid sequence includes cell-free in vitro transcription or translation systems.

In an embodiment, a composition or pharmaceutical composition comprising a con-modified nucleic acid sequence, e.g., when administered to a cell, tissue or subject, results in a product, e.g., a protein, polypeptide or RNA which can be translated into a protein of polypeptide. In embodiments, a composition or pharmaceutical composition comprising a con-modified nucleic acid sequence is contacted with a cell or tissue, or administered to a subject in need thereof, in an amount and for a time sufficient to modulate a production parameter (e.g., an expression parameter and/or a signaling parameter).

In embodiments, a composition or pharmaceutical composition comprising a con-modified nucleic acid sequence is contacted with a cell or tissue, or administered to a subject in need thereof, in an amount and for a time sufficient to modulate (increase or decrease) one or more of the following expression parameters:

(a) protein translation;

(b) expression level (e.g., of polypeptide or protein, or mRNA);

(c) post-translational modification of polypeptide or protein;

(d) folding (e.g., of polypeptide or protein, or mRNA),

(e) structure (e.g., of polypeptide or protein, or mRNA),

(f) transduction (e.g., of polypeptide or protein),

(g) compartmentalization (e.g., of polypeptide or protein, or mRNA),

(h) incorporation (e.g., of polypeptide or protein, or mRNA) into a supermolecular structure, e.g., incorporation into a membrane, proteasome, or ribosome,

(i) incorporation into a multimeric polypeptide, e.g., a homo or heterodimer, and/or

(j) stability.

In embodiments, a composition or pharmaceutical composition comprising a con-modified nucleic acid sequence is contacted with a cell or tissue, or administered to a subject in need thereof, in an amount and for a time sufficient to modulate (increase or decrease) one or more of the following signaling parameters:

(1) modulation of a signaling pathway, e.g., a cellular signaling pathway which is downstream or upstream of the protein encoded by the con-rare codon gene;

(2) cell fate modulation;

(3) ribosome occupancy modulation;

(4) protein translation modulation;

(5) mRNA stability modulation;

(6) protein folding and structure modulation;

(7) protein transduction or compartmentalization modulation; and/or

(8) protein stability modulation.

A production parameter (e.g., an expression parameter and/or a signaling parameter) may be modulated, e.g., by at least 5% (e.g., at least 10%, 15%, 20%, 25%, 30%, 40%. 50%. 60%. 70%, 80%, 90%, 100%, 150%, 200% or more) compared to a production parameter of a reference nucleic acid sequence, e.g., parental, wildtype or conventionally optimized nucleic acid sequence with which said subject, cell or tissue is contacted with.

A con-modified nucleic acid sequence described herein can result in the production of a product, e.g., a protein, polypeptide or RNA (e.g., an RNA that can be translated into a polypeptide). In an embodiment, a product of a con-modified nucleic acid sequence has an altered production parameter, e.g., as compared to the corresponding production parameter of a product of a reference sequence, e.g., parental sequence. The resultant product of the con-modified nucleic acid sequence can be used for treating a disease or disorder described herein and/or for ameliorating a symptom of a disease or disorder, e.g., as described herein.

For example, the disclosure provides a method of delivering to a subject in need thereof, a product (e.g., polypeptide or RNA) of a contextually-modified (“con-modified”) nucleic acid sequence, comprising providing, e.g., administering, to the subject a product (e.g., polypeptide or RNA) of a con-modified nucleic acid sequence. In an embodiment, the product comprises an RNA, e.g., mRNA or an RNA that can be translated. In another embodiment, the product comprises polypeptide.

### Con-Modified Nucleic Acid Sequence Quality Control and Production Assessment

A con-modified nucleic acid sequence or a con-modified nucleic acid composition, e.g., a pharmaceutical composition, produced by any of the methods disclosed herein can be assessed for a characteristic associated with the con-modified nucleic acid sequence or preparation thereof, such as purity, host cell protein or DNA content, endotoxin level, sterility, structure, or functional activity of the con-modified nucleic acid sequence. Any of the above-mentioned characteristics can be evaluated by providing a value for the characteristic, e.g., by evaluating or testing the con-modified nucleic acid sequence, the con-modified nucleic acid sequence composition, or an intermediate in the production of the con-modified nucleic acid sequence composition. The value can also be compared with a standard or a reference value. Responsive to the evaluation, the con-modified nucleic acid sequence composition can be classified, e.g., as ready for release, meets production standard for human trials, complies with ISO standards, complies with cGMP standards, or complies with other pharmaceutical standards. Responsive to the evaluation, the con-modified nucleic acid sequence composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the con-modified nucleic acid sequence composition. For example, the con-modified nucleic acid sequence composition can be modulated, processed or re-processed to (i) increase the purity of the con-modified nucleic acid sequence composition; (ii) decrease the amount of HCP in the composition; (iii) decrease the amount of DNA in the composition; (iv) decrease the amount of fragments in the composition; (v) decrease the amount of endotoxins in the composition; (vi) increase the level and/or activity of a product of the con-modified nucleic acid sequence (e.g., a protein, polypeptide or RNA); or (vii) inactivate or remove any viral contaminants present in the composition, e.g., by reducing the pH of the composition or by filtration.

### Con-Modified Nucleic Acid Sequence Administration

A con-modified nucleic acid sequence, a con-modified nucleic acid sequence composition or a con-modified nucleic acid sequence pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, intratumoral or epidural.

### Vectors and Carriers

In some embodiments the con-modified nucleic acid sequence, or con-modified nucleic acid sequence composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A con-modified nucleic acid sequence, a con-modified nucleic acid sequence composition or a pharmaceutical con-modified nucleic acid sequence composition described herein may comprise, may be formulated with, or may be delivered in, a carrier.

### Viral Vectors

The carrier may be a viral vector (e.g., a viral vector comprising a sequence encoding a con-modified nucleic acid sequence). The viral vector may be administered to a cell or to a subject (e.g., a human subject or animal model) to deliver a con-modified nucleic acid sequence, a con-modified nucleic acid sequence composition or a pharmaceutical con-modified nucleic acid sequence composition. A viral vector may be systemically or locally administered (e.g., injected).

Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A con-modified nucleic acid sequence, a con-modified nucleic acid sequence composition or a pharmaceutical con-modified nucleic acid sequence composition described herein can be administered to a cell in a vesicle or other membrane-based carrier.

In embodiments, a con-modified nucleic acid sequence, a con-modified nucleic acid sequence composition or a pharmaceutical con-modified nucleic acid sequence composition described herein is administered in or via a cell, vesicle or other membrane-based carrier. In one embodiment, the con-modified nucleic acid sequence, a con-modified nucleic acid sequence composition or a pharmaceutical con-modified nucleic acid sequence composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the con-modified nucleic acid sequence, a con-modified nucleic acid sequence composition or a pharmaceutical con-modified nucleic acid sequence composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the con-modified nucleic acid sequence, a con-modified nucleic acid sequence composition or a pharmaceutical con-modified nucleic acid sequence composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a con-modified nucleic acid sequence, a con-modified nucleic acid sequence composition or a pharmaceutical con-modified nucleic acid sequence composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the con-modified nucleic acid sequence, a con-modified nucleic acid sequence composition or a pharmaceutical con-modified nucleic acid sequence composition described herein.

### Target Cells or Host Cells

A target cell or a host cell is a cell (e.g., a cultured cell) that can be used for expression and/or purification of a con-modified nucleic acid sequence or a product of a con-modified nucleic acid sequence (e.g., a protein, polypeptide or RNA). In an embodiment, a target cell or a host cell comprises a mammalian cell, e.g., a human cell. In an embodiment, a target cell or a host cell comprises a non-mammalian cell, e.g., a yeast cell. In an embodiment, a host cell comprises a HeLa cell, a HEK293T cell (e.g., a Freestyle 293-F cell), a HT-1080 cell, a PER.C6 cell, a HKB-11 cell, a CAP cell, a HuH-7 cell, a BHK 21 cell, an MRC-S cell, a MDCK cell, a VERO cell, a WI-38 cell, or a Chinese Hamster Ovary (CHO) cell. In an embodiment, a target cell or a host cell comprises a cancer cell, e.g., a solid tumor cell (e.g., a breast cancer cell (e.g., a MCF7 cell), a pancreatic cell line (e.g. a MIA PaCa-2 cell), a lung cancer cell, or a prostate cancer cell, or a hematological cancer cell).

In an embodiment, a target cell or a host cell is a cell that can be maintained under conditions that allow for expression of a con-modified nucleic acid sequence or a product of a con-modified nucleic acid sequence (e.g., a protein, polypeptide or RNA).

### Method of Culturing a Host Cell or a Target Cell

A cell, e.g., a host cell or a target cell, can be cultured in a medium that promotes growth, e.g., proliferation or hyperproliferation of the cell. A cell, e.g., a host cell or a target cell, can be cultured in a suitable media, e.g., any of the following media: DMEM, MEM, MEM alpha, RPMI, F-10 media, F-12 media, DMEM/F-12 media, IMDM, Medium 199, Leibovitz L-15, McCoys's 5A, MDCB media, or CMRL media. In an embodiment the media is supplemented with glutamine. In an embodiment, the media is not supplemented with glutamine. In an embodiment, a cell is cultured in media that has an excess of nutrients, e.g., is not nutrient limiting. A cell can be cultured in a medium comprising or supplemented with one or a combination of growth factors, cytokines or hormones, e.g., one or a combination of serum (e.g., fetal bovine serum (FBS)), HEPES, fibroblast growth factor (FGFs), epidermal growth factors (EGFs), insulin-like growth factors (IGFs), transforming growth factor beta (TGFb), platelet derived growth factor (PDGFs), hepatocyte growth factor (HGFs), or tumor necrosis factor (TNFs).

A cell, e.g., a host cell or a target cell, can also be cultured under conditions that induce stress, e.g., cellular stress, osmotic stress, translational stress, or oncogenic stress.

A cell, e.g., a host cell or a target cell, can be cultured under nutrient limiting conditions, e.g., the host cell is cultured in media that has a limited amount of one or more nutrients. Examples of nutrients that can be limiting are amino acids, lipids, carbohydrates, hormones, growth factors or vitamins.

A cell, e.g., a host cell or a target cell, can comprise an immortalized cell, e.g., a cell which expresses one or more enzymes involved in immortalization, e.g., TERT. In an embodiment, a cell, e.g., a host cell or a target cell, can be propagated indefinitely.

A cell, e.g., a host cell or a target cell, can be cultured in suspension or as a monolayer. Cell cultures can be performed in a cell culture vessel or a bioreactor. Cell culture vessels include a cell culture dish, plate or flask. Exemplary cell culture vessels include 35 mm, 60 mm, 100 mm, or 150 mm dishes, multi-well plates (e.g., 6-well, 12-well, 24-well, 48-well or 96 well plates), or T-25, T-75 or T-160 flasks.

In an embodiment, a target cell or a host cell can be cultured in a bioreactor. A bioreactor can be, e.g., a continuous flow batch bioreactor, a perfusion bioreactor, a batch process bioreactor or a fed batch bioreactor. A bioreactor can be maintained under conditions sufficient to express the con-modified nucleic acid sequence or a product of the con-modified nucleic acid sequence. The culture conditions can be modulated to optimize yield, purity or structure of the con-modified nucleic acid sequence or a product of the con-modified nucleic acid sequence.

In an embodiment, a bioreactor is maintained under conditions that promote growth of the target cell or host cell, e.g., at a temperature (e.g., 37° C.) and gas concentration (e.g., 5% CO₂) that is permissive for growth of the target cell or host cell. Any suitable bioreactor diameter can be used.

In an embodiment, the bioreactor can have a volume between about 100 mL and about 100 L.

### Method of Modifying a Host Cell or a Target Cell

A cell, e.g., a host cell or a target cell, can be modified to optimize the production of a con-modified nucleic acid sequence or a product of a con-modified nucleic acid sequence e.g., to have optimized RNA or polypeptide yield, purity, structure (e.g., folding), or stability. In an embodiment, a cell, e.g., a host cell or a target cell, can be modified (e.g., using a method described herein), to increase or decrease the expression of a desired molecule, e.g., gene, which optimizes production of a con-modified nucleic acid sequence or a product of a con-modified nucleic acid sequence, e.g., optimizes yield, purity, structure or stability of the RNA or polypeptide product. In an embodiment, a cell, e.g., a host cell or a target cell, can be epigenetically modified, e.g., using a method described herein, to increase or decrease the expression of a desired gene, which optimizes production of a con-modified nucleic acid sequence or a product of a con-modified nucleic acid sequence.

In an embodiment, a cell, e.g., a host cell or a target cell, can be modified by: transfection (e.g., transient transfection or stable transfection); transduction (e.g., viral transduction, e.g., lentiviral, adenoviral or retroviral transduction); electroporation; lipid-based delivery of an agent (e.g., liposomes), nanoparticle based delivery of an agent; or other methods known in the art. In an embodiment, an agent comprises a con-modified nucleic acid sequence or a product of a con-modified nucleic acid sequence.

In an embodiment, a cell, e.g., a host cell or a target cell, can be modified to increase the expression of, e.g., overexpress, a desired molecule, e.g., a gene (e.g., an oncogene, or a gene involved in RNA or protein modulation. Exemplary methods of increasing the expression of a gene include: (a) contacting the host cell with a nucleic acid (e.g., DNA, or RNA) encoding the gene; (b) contacting the host cell with a peptide that expresses the target protein; (c) contacting the host cell with a molecule (e.g., a small RNA (e.g., a micro RNA, or a small interfering RNA) or a low molecular weight compound) that modulates, e.g., increases the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of a negative regulator of the target gene. In an embodiment, a nucleic acid encoding the gene, or a plasmid containing a nucleic acid encoding the gene can be introduced into the host cell by transfection or electroporation. In an embodiment, a nucleic acid encoding a gene can be introduced into the host cell by contacting the host cell with a virus (e.g., a lentivirus, adenovirus or retrovirus) expressing the gene.

In an embodiment, a cell, e.g., a host cell or a target cell, can be modified to decrease the expression of, e.g., minimize the expression, of a desired molecule, e.g., a gene (e.g., a tumor suppressor, or a gene involved in RNA or protein modulation). Exemplary methods of decreasing the expression of a gene include: (a) contacting the host cell with a nucleic acid (e.g., DNA, or RNA) encoding an inhibitor of the gene (e.g., a dominant negative variant or a negative regulator of the gene or protein encoded by the gene); (b) contacting the host cell with a peptide that inhibits the target protein; (c) contacting the host cell with a molecule (e.g., a small RNA (e.g., a micro RNA, or a small interfering RNA) or a low molecular weight compound) that modulates, e.g., inhibits the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of the target gene. In an embodiment, a nucleic acid encoding an inhibitor of the gene, or a plasmid containing a nucleic acid encoding an inhibitor of the gene can be introduced into the host cell by transfection or electroporation. In an embodiment, a nucleic acid encoding an inhibitor of the gene can be introduced into the cell, e.g., a host cell or a target cell, by contacting the cell, e.g., a host cell or a target cell, with a virus (e.g., a lentivirus, adenovirus or retrovirus) expressing the inhibitor of the gene.

All references and publications cited herein are hereby incorporated by reference.

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### EXAMPLES

Table of Contents for Examples

| | |
|---|---|
| Example | Title |

Con-rare codon identification

| | |
|---|---|
| 1a | Quantitative tRNA profiling by Oxford Nanopore sequencing |
| 1b | Quantitative tRNA profiling by next generation sequencing |
| 2 | Quantification of protein expression levels across cell lines or tissue types |
| 3 | Evaluation of contextual rarity and identification of contextually rare codons |
| 4 | Identification of a nucleic acid sequence having con-rare codons (A) |
| 5 | Identification of a nucleic acid sequence having con-rare codons (B) |
| 6 | Exemplary nucleic acid sequence having con-rare codons |
| 7 | Exemplary computational pipeline for codon modifying a nucleic acid sequence |

Method of making a contextually modified nucleic acid sequence

| | |
|---|---|
| 8 | Codon modification of a nucleic acid sequence in a mammalian production host cell |
| 9 | Codon modification of a nucleic acid sequence in a cell free system |

### Example 1: Quantitative tRNA Profiling by Oxford Nanopore Sequencing

This Example describes the quantification of tRNA levels in a cell line or tissue type, which is useful for identifying con-rare codons and candidate con-rare codons.

Transfer RNA levels are determined using Oxford Nanopore direct RNA sequencing, as previously described in Sadaoka et al., Nature Communications (2019) 10, 754.

Briefly, cells transfected with a tRNA molecule are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 30 minutes. The solution is neutralized by the addition of an equal volume of 100 mM Na-acetate/acetic acid (pH 4.8) and 100 mM NaCl, followed by ethanol precipitation. Deacylated sRNA is dissolved in water, and its integrity verified by agarose gel electrophoresis. Deacylated sRNA is then polyadenylated using yeast poly(A) tailing kit per manufacturer's instructions to generate a sRNA polyadenylated pool. Following polyadenylation, a reverse transcription reaction is performed to generate cDNA using SuperScript III Reverse Transcriptase (Thermo Fisher Scientific) or a thermostable group II intron RT (TGIRT, InGex LLC) that is less sensitive to RNA structure and modifications. A sequencing adapter is ligated onto the cDNA mixture by incubating the cDNA mixture with RNA adapter, T4 ligase and ligation buffer following the standard protocol for Oxford Nanopore resulting in a cDNA library. Nanopore sequencing is then performed on the libraries and the sequences are mapped to a genomic database, in this example to the genomic tRNA database, GtRNAdb. The methods described in this example can be adopted for use to evaluate the tRNA pool across cell lines or tissue types.

### Example 1b: Quantitative tRNA Profiling by Next Generation Sequencing

This Example describes the quantification of tRNA levels in a cell line or tissue type. Transfer RNA levels are determined using next generation sequencing, as previously described in Pinkard et al., Nature Communications (2020) 11, 4104.

Briefly, cells transfected with a tRNA molecule are lysed and total RNA is purified using a method such as phenol chloroform. RNAs smaller than 200 nucleotides are separated from the lysate using a small RNA isolation kit per manufacturer's instructions, to generate a small RNA (sRNA) fraction.

The sRNA fraction is de-acylated using 100 mM Tris-HCl (pH 9.0) at 37° C. for 45 minutes. The solution is neutralized by the addition of an equal volume of 100 mM Na-acetate/acetic acid (pH 4.8) and 100 mM NaCl, followed by ethanol precipitation. Deacylated sRNA is splint ligated in a reaction with 3′ adapter, a mix of 4 splint strands and annealing buffer at 37° C. for 15 minutes followed by addition of a RNL2 ligase reaction buffer mix at 37° C. for 1 h and then at at 4° C. for 1 hr. The deacylated and splint ligated sRNA is precipitated using a method such as phenol chloroform extraction.

The deacylated and splint ligated sRNA is reverse transcribed using an RT enzyme such as Superscript IV at 55° C. for 1 hr. The reaction product is desalted in a micro bio0sepin P30 according to manufacturer directions and sample is run on a denaturing polyacrylamide gel. Gel band from 65-200 nt was excised, and sRNA was extracted. The sRNA was circularized using a circligase and purified. The purified circularized RNA was PCR amplified and product run on a e-gel ex. Bands from 100-250 nt were excised and purified using qiaquick gel extraction kit according to manufacturer directions and RNA was precipitated. Next generation sequencing is then performed on the libraries and the sequences are mapped to a genomic database, in this example to the genomic tRNA database, GtRNAdb. The methods described in this example can be adopted for use to evaluate the tRNA pool across cell lines or tissue types.

### Example 2: Quantification of Protein Expression Levels Across Cell Lines or Tissue Types

This Example describes the quantification of protein expression levels across cell lines or tissue types, which is useful in part for identifying con-rare codons and candidate con-rare codons.

### Cell Culture/Sample Preparation

The protein expression levels are monitored using SILAC based mass-spectrometry proteomics, as previously described in Geiger et al., Molecular and Cellular Proteomics (2012) 10, 754.

Briefly, populations of cells are cultured either in media containing isotope-labeled amino acids, such as Lys8 (e.g., 13C615N2-lysine) and Arg10 (e.g., 13C615N4-arginine); or in media containing natural amino acids. The media is further supplemented with 10% dialyzed serum. Cell cultured in media containing isotope-labeled amino acids incorporate the isotope-labeled amino acids into all of the proteins translated after incubation with said isotope-labeled amino acids. For example, all peptides containing a single arginine will be 6 Da heavier in cells cultured in the presence of instead of isotope-labeled amino acid compared to cells cultured with natural amino acids. Cultured are lysed and sonicated. Cell lysates (e.g., about 100 g) are diluted in 8 M urea in 0.1 M Tris-HCl followed by protein digestion with trypsin according to the FASP protocol (Wisniewski, J. R., et al. (2009) Universal sample preparation method for proteome analysis. Nat. Methods 6, 359-362). After an overnight digestion, peptides are eluted from the filters with 25 mM ammonium bicarbonate buffer. From each sample, about 40 ug of peptides are separated into six fractions by strong anion exchange as described previously (Wisniewski, J. R., et al. (2009) Combination of FASP and StageTip-based fractionation allows in-depth analysis of the hippocampal membrane proteome. J. Proteome Res. 8, 5674-5678).

Eluted peptides are concentrated and purified on C18 StageTips, e.g., as described in Rappsilber et al., Nature Protocols (2007).

### LC-MS/MS Analysis

Peptides are separated by reverse-phase chromatography using a nano-flow HPLC (Easy nanoLC, Thermo Fisher Scientific). The high performance liquid chromatography (HPLC) is coupled to an LTQ-Orbitrap Velos mass spectrometer (Thermo Fisher Scientific). Peptides are loaded onto the column with buffer A (0.5% acetic acid) and eluted with a 200 min linear gradient from 2 to 30% buffer B (80% acetonitrile, 0.5% acetic acid). After the gradient the column is washed with 90% buffer B and re-equilibrated with buffer A.

Mass spectra are acquired in a data-dependent manner, with an automatic switch between MS and MS/MS scans using a top 10 method. MS spectra are acquired in the Orbitrap analyzer, with a mass range of 300-1650 Th and a target value of 106 ions. Peptide fragmentation is performed with the HCD method and MS/MS spectra is acquired in the Orbitrap analyzer and with a target value of 40,000 ions. Ion selection threshold is set to 5000 counts. Two of the data sets are acquired with a high field Orbitrap cell in which the resolution is 60,000 instead of 30,000 (at 400 m/z) for the MS scans. In the first of the two replicates with the high field Orbitrap MS/MS scans are acquired with 15,000 resolution, and in the second with 7500 resolution, which is the same as in the standard Orbitrap, but with shorter transients.

### Data Analysis

Raw MS files are analyzed by MaxQuant using standard metrics, e.g., as described in Table 2 of Tyanova S et al. (2016) Nat. Protocols 11(12) pp. 2301-19. Categorical annotation is supplied in the form of Gene Ontology (GO) biological process, molecular function, and cellular component, the TRANSFAC database as well as participation in a KEGG pathway and membership in a protein complex as defined by CORUM.

The methods described in this example can be adopted for use to evaluate the protein expression levels across cell lines or tissue types.

### Example 3: Evaluation of Contextual Rarity and Identification of Contextually Rare Codons

This example describes the method used to determine components of contextual rarity (con-rarity) for con-rare codons or candidate con-rare codons. This method utilizes the cell line or tissue protein expression level determined by proteomics described in Example 2 or taken from literature. This method also utilizes the tRNA profile determined by Nanopore or other tRNA sequencing platform described in the Example 1 or taken from literature.

### Codon Count Per Nucleic Acid Sequence

Using the coding DNA sequence (CDS) defined using National Center for Biotechnology Information (NCBI https://www.ncbi.nlm.nih.gov/) or other database, the protein-coding sequence is segmented into codons and summed per codon to give a codon count per nucleic acid sequence, e.g., gene, for each codon encoded in the protein-coding sequence.

### Normalized Proteome Codon Count

The codon count per nucleic acid sequence, e.g., gene, is then multiplied by the corresponding cell line or tissue protein expression level determined by proteomics to give a cell type normalized proteome codon count across the cell line or tissue.

### Con-Rarity

Con-rarity is a function of normalized proteome codon and the tRNA expression level. In an embodiment, the con-rarity is determined by dividing the normalized proteome codon count by the tRNA expression level determined by Nanopore or other tRNA sequencing experiment. This provides a measure of codon usage that is contextually dependent on the tRNA profile, e.g., tRNA abundance levels. A codon is determined to be contextually rare (con-rare) if the con-rarity meets a reference value, e.g., a pre-determined or pre-selected reference value, e.g., a threshold. In an embodiment, a codon is con-rare if the value of a normalized proteome codon count divided by the tRNA expression level for a particular tRNA meets a pre-determined reference. In an embodiment, the reference value is a value under e.g., 1.5× sigma of the normally fit distribution to that codon frequency. See, for example, FIG. 2.

### Example 4: Identification of a Nucleic Acid Sequence Having Con-Rare Codons (A)

This Example describes the identification of a nucleic acid sequence having con-rare codons or candidates for con-rare codons. Con-rare codons are identified as described in Example 3.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Con-Rare Count Per Nucleic Acid Sequence

Each codon, per nucleic acid sequence, e.g., gene, is classified as a con-rare codon or a con-abundant codon. The counts for all con-rare codons, for each nucleic acid sequence, are summed and normalized to the sequence length.

### Determining a Nucleic Acid Sequence Having Con-Rare Codons

The con-rare codon count is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons if it falls above a reference value, e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 5: Identification of a Nucleic Acid Sequence Having Con-Rare Codons (B)

This Example describes the identification of a nucleic acid sequence having con-rare codons or candidates for con-rare codons. Con-rare codons are identified as described in the Example 3.

### Codon Count Per Nucleic Acid Sequence

Using the coding DNA sequences (CDS) defined using National Center for Biotechnology Information (NCBI https://www.ncbi.nlm.nih.gov/) or other database, all human gene sequences are segmented into codons and summed per codon to give a codon count per nucleic acid sequence, e.g., gene.

### Determining a Nucleic Acid Sequence Having Con-Rare Codons

Each codon, per nucleic acid sequence, e.g., gene, is classified as a con-rare codon or a con-abundant codon. For each con-rare codon, the counts per nucleic acid sequence is fit to a normalized distribution. A nucleic acid sequence that meets a reference value, e.g., a pre-determined reference value, is classified as a nucleic acid sequence having con-rare codons. In an embodiment, a nucleic acid sequence is classified as having con-rare codons, e.g., specified con-rare codons, if it falls e.g., in the upper 3sigma of the normalized distribution. In an embodiment, a nucleic acid sequence having con-rare codons can have one, two, or more (e.g., 3, 4, 5, 6, 7, 8, 9, 10, 20, 30, 50, 100, 200, 500) of the same con-rare codon or different con-rare codons.

### Example 6: Exemplary Nucleic Acid Sequence Having Con-Rare Codons

This Example describes an exemplary nucleic acid sequence having con-rare codons or candidates for con-rare codons.

The GRK2 nucleic acid sequence encodes the GRK2 protein (G-protein coupled receptor kinase 2). The method of Examples 4 or 5 was used to identify the GRK2 nucleic acid sequence as having con-rare codons. The GRK2 nucleic acid sequence has a coding sequence that has con-rare codons AAG and CTG. The AAG codon codes for lysine and the CTG codon codes for leucine. Under certain cellular conditions, the expression of the GRK2 protein can be affected by the frequency of tRNAs corresponding to one or more con-rare codons in the GRK2 nucleic acid sequence, e.g., CUU-tRNA which corresponds to con-rare codon AAG, and/or CAG-tRNA which corresponds to con-rare codon CTG.

### Example 7: Exemplary Computational Pipeline for Codon Modifying a Nucleic Acid Sequence

This Example describes the computational pipeline that can be utilized to codon modify a nucleic acid sequence.

### Mapping Con-Rare Codons

Con-rarity (determined using the method described in Example 3) is read into the algorithm. Con-rare codons are identified as described in Example 3. For example, a codon is determined to be contextually rare (con-rare) if the con-rarity meets a reference value, e.g., a pre-determined or pre-selected reference value, e.g., a threshold. A corresponding contextually abundant (con-abundant) codon is identified as the most contextually frequent codon that encodes the same amino acid as the con-rare codon (e.g., an isoacceptor or an isodecoder). In an embodiment, a con-rare codon can have more than one corresponding con-abundant codon. In an embodiment, the corresponding con-abundant codon can be utilized to replace a con-rare codon.

### Con-Rare Codon Modification

Each sequence to be modified is read in and segmented into codons. Each codon is then evaluated to determine if it is a con-rare codon. If the codon is identified as a con-rare codon, the codon is replaced, e.g., with a corresponding con-abundant codon. A con-abundant codon is a codon other than a con-rare codon. This process can be repeated for two, three, four, or a portion of, or all of the con-rare codons found in the sequence. The resultant con-rare modified sequence (e.g., also referred to as contextually modified nucleic acid sequence) is then outputted.

### Example 8: Codon Modification of a Nucleic Acid Sequence in a Mammalian Production Host Cell

This Example describes the codon modification of a nucleic acid sequence encoding a protein produced in mammalian host cells.

### Plasmid Generation

To generate a plasmid encoding a protein of interest with preferred codon usage, the open reading frame (ORF) of the corresponding nucleic acid sequence encoding the protein of interest, e.g., gene, is processed through a computational pipeline, e.g., as described in Example 7, resulting in a contextually modified ORF (con-modified ORF) nucleic acid sequence. The con-modified ORF is cloned according to the manufacturer's instructions into a mammalian protein expression vector, such as the pcDNA3.1 backbone plasmid containing a RNA polymerase II recruiting promoter, such as the CMV enhancer-promoter, and a luminescence reporter, such as the NanoLuc reporter. To serve as a control, the ORF of the parental sequence (e.g., wildtype sequence, e.g., the sequence from which the con-modified ORF is derived), is similarly cloned into a pcDNA3.1 backbone plasmid containing a RNA polymerase II recruiting promoter, such as the CMV promoter, and a luminescence marker different from one used in the optimized ORF vector, such as the Firefly reporter.

### Transfection

One milligram of each plasmid described above is used to transfect a 1 L culture of suspension-adapted HEK293T cells (Freestyle 293-F cells) at 1×10⁵ cells/mL. Cells are harvested at 24, 48, 72, or 96 hours post-transfection to determine the optimized timepoint for protein expression as determined by Northern blot, or by quantitative PCR (q-PCR).

Quantification of Protein Expression from Con-Modified ORF

Harvested cells are trypsinized, washed and lysed and protein production is determined by fluorescence intensity using a microplate reader at 37° C. λ460/λ565 corresponding to Nanoluc and Firefly, respectively. The ratio of amount of fluorescence is plotted to determine the rate of translation elongation of the parental ORF compared to the con-modified ORF.

### Example 9: Codon Modification of a Nucleic Acid Sequence in a Cell Free System

This Example describes the codon modification of a nucleic acid sequence encoding a protein of interest produced in a cell free system.

mRNA Production

The DNA sequence of the nucleic acid encoding the corresponding protein is processed through a computational pipeline, e.g., as described in Example 7, resulting in a contextually modified nucleic acid sequence. A DNA plasmid containing a bacteriophage T7 promoter followed by the con-modified nucleic acid sequence of interest, followed by a luminescence reporter, such as the NanoLuc reporter, is linearized and transcribed in vitro with T7 RNA polymerase at 37° C. for 45 min, phenol extracted, filtered using a Nuc-trap column, and ethanol precipitated. Similarly, the wildtype nucleic acid sequence of the parental sequence, e.g., the sequence from which the modified nucleic acid sequence is derived, is produced using a different luminescence reporter, such as a Firefly reporter.

### Quantification

A mammalian lysate, such as a rabbit reticulocyte lysate or a HEK293T human cell-derived lysate, is generated as described in (Rakotondrafara, A. M. & Hentze, M. W. Nature Protocols 6, 563-571 (2011)). In this example, 0.1-0.5 ug/uL of mRNA containing the con-modified nucleic acid sequence and parental (e.g., wildtype) nucleic acid sequence is added to the in vitro translation assay lysate. The progress of mRNA translation is monitored by fluorescence increase on a microplate reader at 37° C. using λ460/λ565 with data points collected every 30 seconds over a period of 1 hour. The amount of fluorescence change over time is plotted to determine the rate of translation elongation of the parental nucleic acid sequence compared to the con-modified nucleic acid sequence.

What is claimed is:

1. A method of modifying, e.g., contextually modifying, a nucleic acid sequence, e.g., a nucleic acid sequence comprising an open reading frame (ORF) or coding sequence (CDS), for expression in a target cell or tissue comprising:
a) acquiring the identity of a contextually rare codon (“con-rare codon”) for the nucleic acid sequence in the target cell or tissue;
b)(i) replacing a con-rare codon in the nucleic acid sequence with a contextually abundant (“con-abundant”) codon for the nucleic acid sequence; and/or (ii) replacing a con-abundant codon in the nucleic acid sequence with a con-rare codon for the nucleic acid sequence.
thereby providing a modified nucleic acid sequence which is contextually modified (“con-modified”).

2. A method of modifying, e.g., contextually modifying, a nucleic acid sequence for a target cell or a tissue, comprising:
replacing a contextually rare codon (“con-rare codon”) with a different codon, e.g., a codon that is contextually abundant (“con-abundant”); and/or
replacing a con-abundant codon with a different codon, e.g., a codon that is con-rare,
thereby providing a modified nucleic acid sequence which is contextually modified (“con-modified”).

3. A method of modifying, e.g., contextually modifying, a nucleic acid sequence in a target cell or a tissue, comprising:
(a) acquiring a value for a contextually rare codon (“con-rare codon”), wherein the value is a function of one or more of the following factors, e.g., by evaluating or determining one or more of the following factors:
(1) the sequence of the codon;
(2) the availability of a corresponding tRNA, e.g., charged tRNA, for that con-rare codon in a target cell or tissue, e.g., one or more iso-acceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the tRNA corresponding to the con-rare codon,
(b) replacing the con-rare codon with a different codon, e.g., a codon that is contextually abundant (“con-abundant”).

4. A method of identifying a nucleic acid sequence containing a contextually-rare codon (“con-rare codon”) comprising,
acquiring a value for a con-rare codon in the nucleic acid sequence, wherein the value is a function of one or more of the following factors, e.g., by evaluating or determining one or more of the following factors:
(1) the sequence of the codon;
(2) the availability of a corresponding tRNA, e.g., charged tRNA, for that con-rare codon in a target cell or tissue, e.g., one or more iso-acceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the tRNA corresponding to the con-rare codon,
thereby identifying the con-rare codon in the nucleic acid sequence.

5. A method of making a contextually modified (“con-modified”) nucleic acid, e.g., a DNA or RNA sequence, sequence for a target cell or a tissue, comprising:
synthesizing a nucleic acid sequence comprising a codon that differs in contextual-rareness (“con-rarity”) from the corresponding codon of a reference sequence, e.g., a parental sequence, a starting sequence, a wildtype sequence or a conventionally optimized nucleic acid sequence,
thereby making a con-modified nucleic acid sequence.

6. The method of claim 5, wherein the con-modified nucleic acid sequence comprises one less or one more con-rare codon than a reference sequence.

7. The method of any of the preceding claims, wherein the target cell or tissue is a specific or selected target cell or tissue, e.g., a cell or tissue type in a particular developmental stage; a cell or tissue type in a particular disease state; or a cell present in a particular extracellular milieu.

8. A method of manufacturing a cell comprising a contextually-modified (“con-modified”) nucleic acid sequence, comprising:
forming the con-modified nucleic acid sequence in the cell, e.g., by introducing the con-modified nucleic acid into the cell,
thereby manufacturing a cell comprising a con-modified nucleic acid sequence.

9. The method of claim 8, wherein the cell is homologous to the con-modified nucleic acid sequence.

10. The method of claim 8, wherein the cell is heterologous to the con-modified nucleic acid sequence.

11. A contextually-modified (“con-modified”) nucleic acid sequence.

12. A cell comprising a contextually-modified (“con-modified”) nucleic acid sequence.

13. The con-modified nucleic acid sequence of claim 11, or the cell comprising the con-modified nucleic acid sequence of claim 12, wherein the con-modified nucleic acid sequence comprises DNA.

14. The con-modified nucleic acid sequence of claim 11, or the cell comprising the con-modified nucleic acid sequence of claim 12, wherein the con-modified nucleic acid sequence comprises an RNA, e.g., an RNA that can be translated into a polypeptide, e.g., an mRNA.

15. A method of expressing a nucleic acid sequence or obtaining the product of a nucleic acid sequence, comprising:
providing a contextually-modified (“con-modified”) nucleic acid sequence;
contacting the con-modified nucleic sequence with a cell or a cell-free production system; and
expressing the con-modified nucleic acid sequence,
wherein the con-modified nucleic acid sequence comprises a codon that differs in contextual-rareness (“con-rarity”) from the corresponding codon of a reference sequence, e.g., a parental sequence, a starting sequence, a wildtype sequence or a conventionally optimized nucleic acid sequence,
thereby expressing a nucleic acid sequence or obtaining the product of a nucleic acid sequence.

16. A system for identifying a codon-value for a nucleic acid sequence for expression in a target cell or tissue, comprising:
a computer, e.g., a general purpose computer, configured for:
optionally, acquiring a nucleic acid sequence from the target cell or tissue;
a) acquiring, e.g., calculating, a codon-value which is a function of the contextual-rareness (“con-rarity”) of a sequence-codon in a sequence, wherein the con-rarity of a codon is a function of one or more of the following factors, e.g., by evaluating one or more of the following factors:
(1) the sequence of the codon;
(2) the availability of a corresponding tRNA, e.g., charged tRNA, for that con-rare codon in a target cell or tissue, e.g., one or more isoacceptor tRNA molecules;
(3) the expression profile (or proteomic properties) of the target cell or tissue (e.g., the abundance of expression of other proteins which include the con-rare codon);
(4) the proportion of the tRNAs corresponding to the con-rare codon which are charged; and
(5) the iso-decoder isotype of the t RNA corresponding to the con-rare codon,
b) memorializing, e.g., recording, the codon-value on a medium, e.g., in a machine readable medium,
thereby identifying the codon-value for the nucleic acid sequence.
